(19) United States
(12) Patent Application Publication
(10) Pub. No.: US 2002/0124329 A1
(43) Pub. Date: Sep. 12, 2002

(54) HAIR COLOURING COMPOSITIONS AND THEIR USE

(76) Inventor: DOMINIC PRATT, BERKSHIRE (GB)

Correspondence Address: T DAVID REED, THE PROCTER & GAMBLE COMPANY, 5299 SPRING GROVE AVENUE, CINCINNATI, OH, 45217-1087, US

(21) Appl. No.: 09/424,473

(22) PCT Filed: May 22, 1998
(86) PCT No.: PCT/US98/10503

(30) Foreign Application Priority Data
May 23, 1997 (GB) 9710761.9
May 23, 1997 (GB) 9710754.4
May 23, 1997 (GB) 9710756.9
May 23, 1997 (GB) 9710757.7
May 23, 1997 (GB) 9710759.3

(51) Int. Cl. A61K 7/13

(52) U.S. Cl. 8/405; 8/406; 8/421

(57) ABSTRACT

Novel hair coloring compositions are provided which give improved fade resistance. They comprise (i) one or more developers selected from amino aromatic systems capable of being oxidized and thereafter undergoing a single electrophilic attack, and (ii) one or more couplers selected from (A) phenols and naphthols having an active leaving group in the para position relative to the OH group, (B) 1,3-diketones containing the group (I) in which Z is an active leaving group, and (C) compounds containing the group (II) in which Z is an active leaving group, and X is an active leaving group or a non-leaving substituent, such that in the presence of an oxidizing agent the or each developer reacts with the or each coupler substantially only at the position having the active leaving group Z and, if X is an active leaving group, X, which when applied to hair by the hair switch coloring method described herein and washed 20 times by the washing protocol described herein, gives a value of “E fade, measured as described herein, as follows: (a) when the composition is suitable for delivering a blonde or light brown shade “E fade is not more than 2.5, (b) when the composition is suitable for delivering a red shade, “E fade is not more than 5.0, (c) when the composition is suitable for delivering a black or dark brown shade “E fade is not more than 2.5.

This invention relates to new compositions for colouring hair and to methods of using these compositions in hair colouring processes.

Compositions are well known for providing various colours to hair by dyeing, either for changing a natural hair colour and/or for covering grey hair. Such compositions comprise various aromatic compounds, commonly known as developers (also known as precursors or primary intermediates), together with various other aromatic compounds, commonly known as couplers. These are referred to as oxidative hair colouring agents because they require an oxidising agent for formation of colour.

The developers are generally 1,4-disubstituted benzene compounds, most commonly 1,4-diaminobenzene compounds, and the couplers can also be disubstituted benzene compounds, such as 1,3-disubstituted benzene compounds. The range of structures of couplers is much more varied than that of developers.

In use, the compounds are subjected to oxidising conditions under which the developers and couplers react to form colour. It is generally believed that this occurs by means of a stepwise sequence in which developer molecules are activated by oxidation and react with couplers to form reactive dimers. These then continue reacting to form coloured trimers, which do not react further. It is believed that the monomeric developers and couplers, and to a lesser extent the dimers, diffuse into the hair shaft during the course of the reaction, which is fairly slow. When inside the hair shaft, the dimers react further to form trimers which are too large to diffuse out easily and are thus trapped, colouring the hair.

Standard products normally include several different developers and several different couplers, for instance up to 5 developers and 5 or more couplers. It is generally accepted that 10 to 12 different compounds are required to achieve the full range of colours.

Unfortunately, this system, although effective and commercially successful, has various drawbacks.

Firstly, developers can react with each other as well as with couplers and the reactive dimers can react with developers and couplers. Thus the reaction chemistry is undefined and it is not possible to predict with any precision the compounds which will be present in the hair at the end of the colour-forming reaction. The exact composition of the coloured molecules formed in the hair can vary from process to process according to the prevailing conditions. Therefore the colours eventually obtained can vary between applications.

A more important drawback is that of fading of colour over time. A contributory factor in fading is lack of wash-fastness. The trimeric coloured molecules produced tend to be soluble in water and in other solvents. Consequently they tend to leach out of the hair after repeated washing and applications of, for instance, hairspray and other hair care products. This leads to gradual fading or changing of the applied colour. The action of other factors such as ultraviolet light, combing and perspiration also affects the colour.

This is a problem which has existed with commercial products for several years and which has not yet been solved.

A further problem arises from the fact that the oxidation reaction involves two steps. Thus sufficient oxidising agent must be present in the hair dye composition to induce the two oxidation steps. The presence of large amounts of oxidising agent can have undesirable effects on skin and hair.

GB 1,025,916 discloses certain developers and couplers of different types. It describes developers which are N,N-disubstituted phenylene diamine derivatives. Three classes of coupler are described. Some phenol-based couplers are said to provide a blue colour, some R—CO—CH₂—COR derivatives are said to provide a yellow colour and some pyrazolone derivatives are said to provide a red colour.

These combinations are advantageous in that the developers do not react with themselves and can react with each coupler in only one way, so that the final chemistry of the dye obtained is closely defined and highly predictable.

GB 1,025,916 describes mixing pairs of couplers, for instance red with blue, blue with yellow, etc so as to obtain shades between the colours which would be obtained with either of the couplers used individually.

This document describes various examples of dyeing hair using the disclosed developers and couplers. In some examples developer is applied and left for a period of time, followed by application of coupler, which is also left for a period of time and is then followed by application of oxidising agent.

The majority of the examples describe mixing developer, coupler and hydrogen peroxide as oxidising agent and applying the mixture to the hair. This is then left for a period of time, normally 20 minutes, and the hair rinsed. This latter method is the standard method of applying currently commercially available hair dyes containing oxidative hair colouring agents. These are normally supplied in a package containing two bottles. One contains developers and couplers and the other contains oxidising agent. These are mixed before application of the mixture to the hair.

We have found that this latter method when applied as described in GB 1,025,916 leads to very poor fade resistance and wash fastness. The applied colouring materials tend to wash out of the hair very rapidly.

We are not aware that a system of the type described in GB 1,025,916 has ever been commercialised.

According to a first aspect of the invention we provide a hair colouring composition comprising

(i) one or more developers selected from amino aromatic systems capable of being oxidised and thereafter undergoing a single electrophilic attack, and

(ii) one or more couplers selected from

(A) phenols and naphthols having an active leaving group in the Para position, relative to the hydroxyl group,

(B) 1,3-diketones containing the group

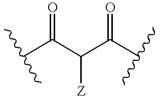

in which Z is an active leaving group, and

(C) compounds containing the group

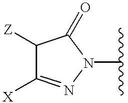

in which Z is an active leaving group, and X is an active leaving group or a non-leaving substituent,

such that in the presence of an oxidising agent the or each developer reacts with the or each coupler substantially only at the position having the active leaving group Z and, if X is an active leaving group, X,

which when applied to damaged hair by the hair switch colouring method described herein and washed 20 times by the washing protocol described herein, gives a value of ΔE fade, measured as described herein, as follows:

(a) when the composition is suitable for delivering a blonde or light brown shade, ΔE fade is not more than 2.5

(b) when the composition is suitable for delivering a red shade, ΔE fade is not more than 5.0,

(c) when the composition is suitable for delivering a black or dark brown shade, ΔE fade is not more than 2.5.

In the invention the developer is an amino aromatic compound which has a structure such that it is capable of being oxidised by an oxidising agent. The structure is also such that the oxidised developer is capable of undergoing electrophilic attack by one other molecule. In other words, the structure of the developer is such that it reacts substantially only at one position, which is normally an amine. Suitable developers of this type include aminoaromatic systems in which there is only one primary amine group, at which reaction occurs, other amine and other reactive groups being protected by blocking substituents.

The three defined types of coupler are such that in the presence of an oxidising agent the majority couple with the developer at only one position so as to produce only one resulting coloured dimer. Certain couplers of type (C), having an additional active leaving group X, also react at the X position to give a single resulting coloured trimer. In this case also only one type of final coloured molecule is produced from that coupler. The defined developer also reacts only at one position.

Formation of colour is, we believe, by reaction of one or two developer molecules with one coupler molecule to form a coloured dimer or trimer. The dimers and trimers are not reactive and no further reaction takes place. Consequently the formation of colour is extremely efficient. Further, the coloured molecules formed are very pure. With knowledge of the developer and coupler molecules present in the reaction system it is possible to predict closely and accurately the final combination of coloured molecules, and hence the final overall colour, which will be produced. These are significant advantages in comparison with standard oxidative colouring systems.

In case (a), ΔE fade is preferably not more than 2.0, more preferably not more than 1.5 and in particular not more than 1.0. Preferably the change in hair colour, % ΔE, after 20 washes is less than about 15%, more preferably less than about 12%, most preferably less than about 10% and in particular less than about 8%.

In case (b), ΔE fade is preferably not more than 4.0, more preferably not more than 2.0, most preferably not more than 1.8. Preferably the change in hair colour, % ΔE, after 20 washes is less than about 20%, more preferably less than about 15%, most preferably less than about 10%.

In case (c), ΔE fade is preferably not more than 2.0, more preferably not more than 1.5, and especially not more than 1.0. Preferably the change in hair colour, % ΔE, after 20 washes is less than about 4.5%, more preferably less than about 4%, most preferably less than about 3.5%.

We have found surprisingly that not only is it possible to provide compositions having significantly improved fade resistance and wash fastness, as demonstrated by low ΔE fade values, in comparison with those described in GB 1,025,916, it is also possible to obtain significant fade resistance and wash fastness improvements over commercially available products.

In this specification, a blonde or light brown shade is one which has a hue value as defined below in the range of from about 70 to about 110. The initial colour intensity L is greater than about 20 and less than about 95. Preferably it is greater than about 25 and less than about 90.

A red shade as defined herein is one which has a hue value of from about 25 to about 70, preferably from about 30 to about 65, most preferably from about 35 to about 60. The initial colour intensity L is greater than about 10 and less than about 70. Preferably it is greater than about 15 and less than about 65, more preferably greater than about 20 and less than about 60.

In this specification a brown or black shade will have a hue value of less than about 25, preferably less than about 20 and the initial colour intensity is preferably greater than about 1 and less than about 50, more preferably greater than about 5 and less than about 45.

In this specification the fade values discussed above are measured using the protocol discussed herein on damaged hair, ie hair that has been permed, bleached, and/or previously coloured. Normally it has been permed and bleached as described below. The hair tested is preferably yak hair.

The improved values of ΔE of the invention have not been previously achievable on damaged hair. The invention provides not only the improved values of ΔE and thus improved wash fastness, but additionally provides a high degree of control of the final chemistry and colour of the colouring materials in comparison with standard oxidative colouring systems.

In general it is found that commercially available prior art systems show less fading from undamaged hair than from damaged hair, which tends to be more porous. Thus the fade from the damaged tips of hair which is coloured is greater than fade from the undamaged roots, leading eventually to uneven coloration. The formulations of GB 1,025,916 tend to give high fade from both damaged and undamaged hair. In the invention we obtain much more even, low fading. Thus preferably when a composition of the invention is used to colour both damaged and undamaged hair, the fading on the damaged hair is preferably not more than twice that on the undamaged hair (measured as ΔE fade), preferably not more than 175%, more preferably not more than 150% of the fading on the undamaged hair. The undamaged hair used is preferably undamaged (virgin) yak hair.

Some suitable coupler molecules (A), (B) and (C) are known from the field of photography, as are some suitable developers. When they have reacted with a developer molecule the couplers (A) give a cyan colour, the couplers (B) give a yellow colour and the couplers (C) give a magenta colour.

Each coupler contains a moiety of a specific formula which is such that it has an active leaving group Z at a defined site. By an “active leaving group” we mean any group which can be removed (under the conditions prevailing during the hair-colouring process) so that the developer reacts at that position in the coupler molecule. The bond formed between the coupler and developer molecule is thus formed at the site of the active leaving group. Examples of active leaving groups are H, PhO, Cl, Br, alkoxy (RO) such as phenoxy PhO, and RS— in which R is alkyl or aryl, but any leaving group which leaves during the reaction so as to allow coupling between developer and coupler is suitable.

If X is an active leaving group it may be any of those listed above for Z.

Couplers (A) give a cyan colour. The particular shade or intensity of colour can be varied by varying the substituents of the phenol or naphthol molecule. It has an active leaving group para to the OH group. This may be an active proton, ie the aromatic ring is unsubstituted in the para position and other substituents on the ring are not such as to reduce the reactivity at this position.

Generally couplers (A) have the formula I, as follows:

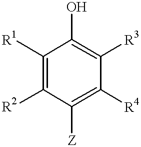

in which Z is H or another active leaving group. Preferably Z is H.

R¹, R², R³ and R⁴ are, independently, H, OH, —CO₂H, —CO₂R, F, Cl, Br, —CN, —NO₂—, CF₃, cycloalkyl, alkenyl, cycloalkenyl, aryl, alkaryl, aralkyl, —NH₂, —NHR, —NHCOR, —NR_{2,} —NHCOR, —R′NHCOR, —CONHR, R′CONHR, —R′OH, —SO₂R, SO₂NHR, —R′SO₂R, —R′SO₂NHR, —SO₃H, —OR, —R′OR or —COR, in any of which R is H, alkyl, cycloalkyl, alkenyl, cycloalkenyl, aryl, alkaryl or aralkyl, and RI is alkylene, alkenylene, cycloalkylene, cycloalkenylene, arylene, alkarylene or aralkylene, or substituted versions of any of these. Additionally, R¹ and R² may together form a substituted or unsubstituted cycloalkyl, cycloalkenyl or aryl group. Substituting groups include OH, —OR, Cl, Br, F, —CO₂H, —CO₂R, —NH₂ and —COR.

In this specification, unless otherwise stated alkyl and alkenyl are usually C₁₋₈, often C₁₋₄, cycloalkyl and cycloalkenyl are usually C₅₋₈, often C₆, aryl or ar- is usually phenyl or naphthyl and the alk-moiety in alkaryl is usually C₁₋₆, often C₁₋₄.

For coupler (A) it is preferred that when R¹, R², R³ or R⁴ is alkyl it is selected from methyl, ethyl, n-propyl, i-propyl and t-butyl. When R is alkyl it is preferably one of these groups and when R′ is alkylene it is preferably derived from one of these groups.

Substituents R¹ and R² may form a second benzene ring, so that coupler (A) is a naphthol derivative of the formula II, as follows:

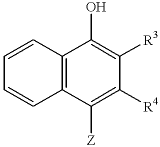

In this case R³ and R⁴ are preferably H and the developer is α-naphthol.

In other suitable couplers (A) of formula II, R⁴ is H and R³ is

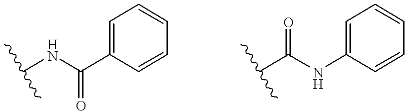

Suitable couplers (A) thus have the formula III or IV, as follows:

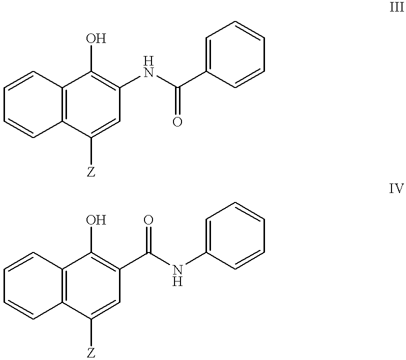

Coupler (A) can be a naphthol having no solubilising substituents (other than Z), in particular no —COOH or —OH substituents. Naphthols are preferably unsubstituted.

We find that couplers of the formula II, in particular when R³ and R⁴ are H, and especially when Z is H, have a particularly advantageous combination of properties for improving wash fastness whilst allowing rapid colouring, when the hair to be coloured has been damaged, for instance by perming or bleaching. We believe this is because their molecules have a structure such that as monomers they are small enough to diffuse easily into the hair shaft (which, when damaged, is rather porous) but as dimers they are trapped within the hair shaft. Further, their water-solubility is low enough that they are not easily washed out during subsequent hair treatment processes.

Preferred couplers have the formula I in which R¹, R², R³ and R⁴ are independently selected from OH, H, methyl, ethyl, n-propyl, i-propyl, t-butyl, NH₂, —CO₂H, and —COR. In these preferred couplers (A) Z is H.

A preferred coupler of this preferred type is 3-amino phenol.

We find that these preferred couplers show particularly good performance on damaged hair. They demonstrate good colour uptake and good wash fastness.

In all of the above formulae Z is any active leaving group. Suitable examples are H, PhO, Cl and Br but any other groups which react similarly (under the conditions of the hair-dyeing reaction) may be used. If Z is PhO, Cl or Br the reactivity of the coupler can tend to be increased in comparison with couplers in which Z is H.

In any of the above formulae the defined groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂ NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different. If phenyl groups are substituted, mono substitution is preferred. Preferably the groups are unsubstituted unless otherwise stated.

Yellow couplers (B) contain the 1,3-diketone group

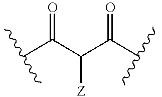

in which Z is an active leaving group. Generally, they have the formula V as follows:

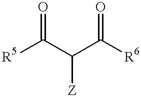

in which R⁵ and R⁶ are, independently, H, alkyl, cycloalkyl, alkenyl, cycloalkenyl, aryl, alkaryl, aralkyl, —R′NHCOR, —R′CONHR, —ROH, —R′SO₂R, —R′CO₂NHR, —NHCOR, —NR₂, —NHR, —NH₂, —R′OR and —OR. In these groups R can be H, alkyl, cycloalkyl, alkenyl, cycloalkenyl, aryl, alkaryl or aralkyl and RI is alkylene, cycloalkylene, alkenylene, cycloalkenylene, arylene, alkarylene or aralkylene. Substituted versions of any of these can be used. Suitable substituents include OH, —OR, Cl, Br, F, —CO₂H, —CO₂R, —NH₂ and —COR.

In some preferred formulae, at least one of R⁵ and R⁶ contains an aryl group.

Some couplers (B) are of the formula VI as follows:

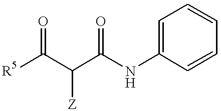

In this formula R⁵ can be for instance methyl, phenyl, t-butyl or N(CH₃)CH₂CH₂OH. When R⁵ is tertiary butyl advantages arise from the fact that the coloured dimer produced has particularly good resistance to breakdown by light. R⁵ may also be phenyl. In formula VI it can be preferred that the N-phenyl does not contain solubilising substituents. In particular it can be preferred that it does not contain —COOH or —OH substituents. Preferably the N-phenyl is unsubstituted. In formulae in which R⁵ is also phenyl it can be preferred that the R⁵ phenyl is free of solubilising substituents, in particular free of —COOH and —OH substituents and is most preferably unsubstituted.

We find that couplers of the formula VI have a particularly advantageous combination of properties for improving wash fastness whilst allowing rapid colouring, in particular for damaged hair. We believe this is because their molecules have a structure such that as monomers they are small enough to diffuse easily into the porous, damaged hair shaft but as dimers they are trapped within the hair shaft. Further, their solubility is such that they are not easily washed out during subsequent hair treatment processes.

In other preferred couplers (B) R⁵ is methyl, ethyl, n-propyl, i-propyl, t-butyl or phenyl (especially methyl) and R⁶ is NR₂ in which the R groups are the same or different and can be R as discussed above, in particular methyl, ethyl, n-propyl, i-propyl, t-butyl or phenyl (especially ethyl).

In other suitable preferred couplers (B) R⁵ and R⁶ are, independently, short chain (C₁₋₄) alkyl such as methyl, ethyl, i-propyl, n-propyl or t-butyl or short chain (C₁₋₄) alkoxy, such as methoxy or ethoxy. In particular, R⁵ is C₁₋₄ alkyl (especially methyl) and R⁶ is C₁₋₄ alkyl (especially methyl) or C₁₋₄ alkoxy (especially methoxy).

In these formulae alkyl groups can advantageously be hydroxylated, to produce for instance hydroxymethyl (usually 2-hydroxyethyl), hydroxyethyl, hydroxypropyl or hydroxybutyl.

Couplers (B) of these latter types are particularly advantageous for the coloration of undamaged as well as damaged hair. On undamaged hair they show fast colour uptake without loss of wash fastness. They also show good wash fastness on damaged hair.

In formulae V and VI, Z may be any of the leaving groups indicated for Z in coupler (A) above. Preferably Z is H.

In any of the above formulae the defined groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂ NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different. If phenyl groups are substituted, mono substitution is preferred. Preferably the groups are unsubstituted unless otherwise stated.

Couplers (C) are pyrazolone derivatives, that is they contain the group

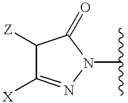

in which Z is an active leaving group and X is an active leaving group or a non-leaving substituent.

Normally X is a non-leaving substituent and they are of the formula VII, as follows:

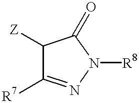

in which R⁷ may be H, —OH, —CO₂H, —CO₂R, F, Cl, Br, —CN, —NO₂, CF₃, alkyl, cycloalkyl, alkenyl, cycloalkenyl, aryl, alkaryl, aralkyl, —NH₂, —NHR, —NR₂, —NHCOR, —R′NHCOR, —CONHR, —R′CONHR, —R′OH, —SO₂R, —SO₂NHR, —R′SO₂R, —R′SO₂NHR, —SO₃H, —OR, —R′OR or —COR. R⁸ can be H, alkyl, alkenyl, cycloalkyl, cycloalkenyl, aryl, alkaryl, aralkyl, —R′NHCOR, —R′CONHR, —R′OH, —R′SO₂R, —R′SO₂NHR or —R′OR. R is H, alkyl, cycloalkyl, alkenyl, cycloalkenyl, aryl, alkaryl or aralkyl, and R′ is alkylene, cycloalkylene, alkenylene, cycloalkenylene, arylene, alkarylene or aralkylene (or substituted versions of any of these). R⁷ and R⁸ may for instance be alkyl, cycloalkyl, alkenyl, cycloalkenyl, aryl, alkaryl, aralkyl, R′NHCOR, R′CONHR, SO₂R, SO₂NHR, R′SO₂R or R′SO₂NHR. Suitable substituting groups include OH, —OR, Cl, Br, F, —CO₂H, —CO₂R, —NH₂, and —COR.

For instance R can be H or methyl. It may alternatively be —NHR or —NHCOR in which R is alkyl, cycloalkyl, alkenyl, cycloalkenyl, aryl, alkaryl or aralkyl.

Advantageously R⁷ is H, lower (C₁₋₄) alkyl such as methyl, ethyl, n-propyl, i-propyl or t-butyl, or substituted or unsubstituted phenyl, in particular H, methyl or methylphenyl.

R⁸ is advantageously H, lower (C₁₋₄) alkyl such as methyl, ethyl, n-propyl, i-propyl or t-butyl, or substituted or unsubstituted phenyl. When it is phenyl, it can be preferred that it is a phenyl free of solubilising substituents, in particular —COOH and —OH substituents. m-SO₃H and p-SO₃H substituents can be used. When R⁸ is phenyl it can preferably be unsubstituted. R⁸ can also preferably be H, phenyl or methyl.

We find that couplers of these latter formulae have a particularly advantageous combination of properties for improving wash fastness whilst allowing rapid colouring, especially on undamaged hair. We believe this is because their molecules have a structure such that as monomers they are small enough to diffuse easily into the undamaged hair shaft but as dimers they are trapped within the hair shaft. Further, their solubility is such that they are not easily washed out during subsequent hair treatment processes.

Suitable couplers (C) have the formula VIII, as follows:

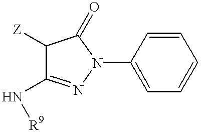

in which R⁹ is preferably

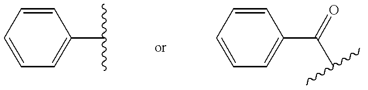

In formulae VII and VIII, Z may be any of the leaving groups indicated for Z in couplers (A) and (B) above.

In any of the above formulae the listed groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂, NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different. If phenyl groups are substituted, mono substitution is preferred. Preferably groups are unsubstituted unless otherwise stated.

Specific examples of couplers (A) include α-naphthol, 3-aminophenol and the compounds having the following structural formulae:

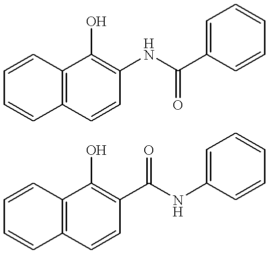

Specific examples of couplers (B) include benzoylacetanilide, acetoacetanilide, N,N-diethyl and N,N-dimethyl acetoacetamide and the compounds of the formulae

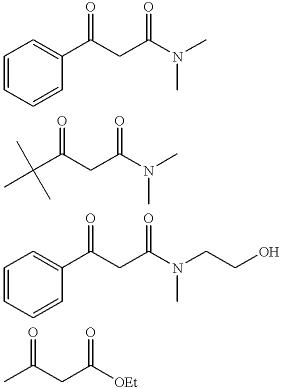

Specific examples of couplers (C) include the pyrazolone of structural formula:

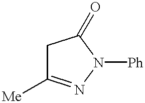

and compounds having the same formula except that Ph is replaced by H or methyl and/or Me is replaced by H, and compounds having the following structural formulae:

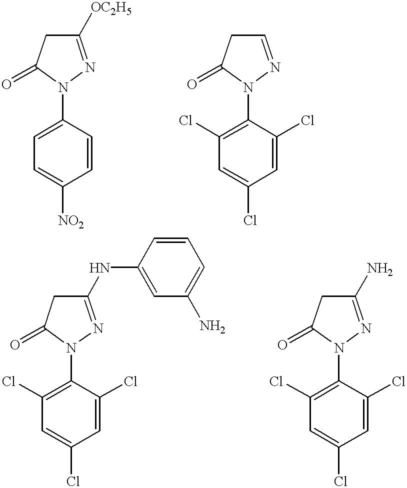

Any of the couplers discussed above may also be used in the salt form, for instance sulphate, phosphate and hydrochloride, particularly sulphate or hydrochloride.

Compounds containing free amine groups are preferably used in the form of their salt. The salt form of such compounds forms a powder and is often more stable than the free base form.

The developer is an amino aromatic compound capable of being oxidised and undergoing a single electrophilic attack in the oxidised state. For instance it may be an aromatic system containing a single primary amine substituent.

The developer is such that it reacts substantially at only one position (normally the amine position). In some cases the structure of the developer may be such that it is possible that it reacts with other developer molecules, but it reacts preferentially with coupler molecules. Preferably the structure of the developer is such that it undergoes substantially no reaction with other developer molecules.

Suitable developers include o-nitro and p-nitro α-naphthylamines of the formulae

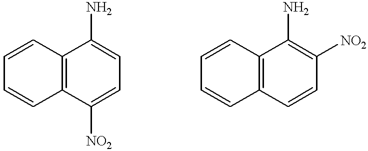

Other suitable developers include o- and p-nitrophenylamines H₂N—Ph—NO₂, N,N-disubstituted o-phenylene diamines and N,N-disubstituted p-phenylene diamines.

The developer can be an N,N-disubstituted p-phenylene diamine. These developers have an amine group protected by disubstitution and react only at the primary amine group. In this case it normally has the formula IX, as follows:

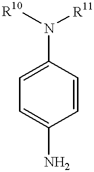

in which R¹⁰ and R¹¹ are each independently H, alkyl, cycloalkyl, alkenyl, cycloalkenyl, aryl, alkaryl, aralkyl, —R′NHCOR, —R′CONHR, —R′OH, —R′SO₂R, —R′SO₂NHR OR —R′OR in which R is alkyl, cycloalkyl, alkenyl, cycloalkenyl, aryl, alkaryl, aralkyl, and R′ is alkylene, cycloalkylene, alkenylene, cycloalkenylene, arylene, alkarylene or aralkylene, or substituted versions of any of these. Suitable substituting groups include OH, —OR, Cl, Br, F, —CO₂H, —CO₂R, —OR and —COR. Alternatively, R¹⁰ and R¹¹ may together form a substituted or unsubstituted cycloalkyl, cycloalkenyl or aryl ring.

Preferably R¹⁰ and R¹¹ are, independently, C₁₋₄ alkyl, preferably —CH₃, —CH₂CH₃ or i-propyl; C₁₋₃ hydroxyalkyl, preferably —CH₂CH₂OH; alkylene alkoxy, preferably ethylmethoxy (—CH₂CH₂OCH₂); or R¹²SO₂NHR¹² or R¹²NHSO₂R¹² in which R¹² is C₁₋₃ alkyl, for instance —CH₂CH₂SO₂NHCH₃ or —CH₂CH₂NHSO₂CH₃.

Particularly preferred developers of the above formula IX are those in which R¹⁰ and R¹¹ are both —CH₂CH₃ or R¹⁰ is —CH₂CH₃ and R¹¹ is —CH₂CH₂NHSO₂CH₃. The latter R¹¹ substituent is believed to contribute to dermatological compatibility. In other suitable developers R¹⁰ is ethyl and R¹¹ is hydroxyethyl; or R¹⁰ is ethyl and R¹¹ is —CH₂CH₂OCH₃; or R¹⁰ is selected from H, methyl, ethyl, and propyl and R¹¹ is selected from methyl, ethyl and propyl.

In general the developer may be selected from compounds having the general formula X:

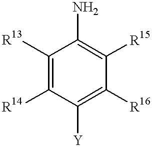

The group Y is a blocking group which ensures that reaction (under the conditions of colouring the hair) takes place only at the primary amine group. The group Y for instance can be —NR¹⁰R¹¹ (as in formula IX above). Other suitable Y groups include —NO₂, —CO₂H, —CO₂R, —COR and OH. R is as defined above for formula IX.

In an alternative developer formula, the blocking group Y is in the ortho position relative to the amino group, giving the following formula XI.

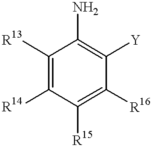

Thus the group Y is positioned so that the developer undergoes only one reaction, at the primary amine group, under the conditions of the reaction.

R¹³, R¹⁴, R¹⁵ and R¹⁶ can each be, independently, any of the groups listed for R¹ to R⁴ above. R¹³ and R¹⁴ together and/or R¹⁵ and R¹⁶ together, may form a substituted or unsubstituted cycloalkyl, cycloalkenyl or aryl ring.

Preferably R¹³ to R¹⁶ are, independently, H, methyl, ethyl, n-propyl, i-propyl, F, Cl, OH, NO₂, —CO₂H, —CO₂R or —COR. In any of the above formulae the listed groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂ NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different. If phenyl groups are substituted, mono substitution is preferred. Preferably the groups are unsubstituted unless otherwise stated.

A preferred developer has the following formula XII, as follows:

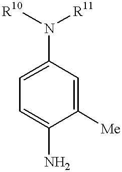

Specific examples of developers of the invention are those of the following structural formulae:

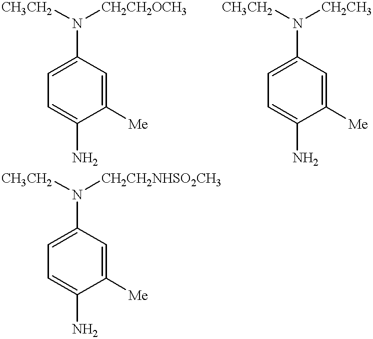

These are suitable especially for colouring of damaged hair.

Additional developers of the invention are:

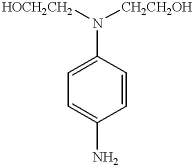

which tends to fade more rapidly than certain others, and

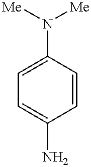

which is highly reactive.

Examples of further preferred developers, which are particularly suitable for coloration of undamaged hair, are 2,6-dichloro-p-aminophenol, 2-chloro-p-aminophenol, 3-chloro-p-aminophenol, 2,3-dichloro-p-aminophenol and 3,5-methyl-p-aminophenol.

The derivatives above include salts, for instance sulphate, phosphate and hydrochloride, particularly sulphate or hydrochloride. Salts are normally formed with the amine groups. The preferred developer in which R¹⁰ is —CH₂CH₃ and R¹¹ is CH₂CH₂NHSO₂CH₃ is often provided in salt form as a stable powder (more stable than the free base form). We have found that this salt forms such that it contains 3 moles of salt molecule to 2 moles of the free base molecule. A further useful salt is the hydrochloride salt of developers such as 2,6-dichloro-p-aminophenol.

We have found that one way of achieving the greatly improved ΔE value is by inclusion of a further component, an antioxidant, which can for instance be a sulphite or a heavy metal chelant. Such systems are described in our copending application number 9710754.4.

An antioxidant can be any material which slows the reaction between the developer, couplers and oxidising agent. It may be selected from for instance sulphites such as sodium sulphite, hydroquinone, sodium bisulphite, sodium metabisulphite, thioglycolic acid, sodium dithionite, erythrobic acid and other mercaptans, ascorbic acid and n-propyl gallate. A preferred antioxidant is sulphite, in particular sodium sulphite.

Certain chelants which slow the reaction can also be used as antioxidants. These include components which act to sequester (chelate or scavenge) heavy metal ions. They may also have calcium and magnesium chelation capacity, but preferentially they show selectivity to binding heavy metal ions such as iron, manganese and copper.

Various sequestering agents, including the amino phosphonates, available as Dequest (RTM) from Monsanto, the nitriloacetates, the hydroxyethyl-ethylene triamines and the like can be suitable. Heavy metal ion sequestrants include organic phosphonates, such as the amino alkylene poly (alkylene phosphonates), alkali metal ethane 1-hydroxy disphosphonates and nitrilo trimethylene phosphonates.

Among the above species are diethylene triamine penta (methylene phosphonate), ethylene diamine tri (methylene phosphonate) hexamethylene diamine tetra (methylene phosphonate) and hydroxy-ethylene 1,1 diphosphonate.

Biodegradable non-phosphorous heavy metal ion sequestrants which can be suitable include nitrilotriacetic acid and polyaminocarboxylic acids such as ethylenediaminotetracetic acid, ethylenetriamine pentaacetic acid, ethylenediamine disuccinic acid, ethylenediamine diglutaric acid, 2-hydroxypropylenediamine disuccinic acid or any salts thereof. Ethylenediamine-N,N′-disuccinic acid (EDDS) (see U.S. Pat. No. 4,704,233), or the alkali metal, alkaline earth metal, ammonium, or substituted ammonium salts thereof, or mixtures thereof, can be used.

Other heavy metal ion sequestrants are iminodiacetic acid derivatives such as 2-hydroxyethyl diacetic acid or glyceryl imino diacetic acid, described in EP-A-317,542 and EP-A-399,133. The iminodiacetic acid-N-2-hydroxypropyl sulfonic acid and aspartic acid N-carboxymethyl N-2-hydroxypropyl-3-sulfonic acid sequestrants described in EP-A-516,102 can be suitable. The β-alanine-N,N′-diacetic acid, aspartic acid-N,N′-diacetic acid, aspartic acid-N-monoacetic acid and iminodisuccinic acid sequestrants described in EP-A-509,382 can also be suitable.

EP-A-476,257 describes amino based sequestrants. EP-A-510,331 describes sequestrants derived from collagen, keratin or casein. EP-A-528,859 describes an alkyl iminodiacetic acid sequestrant. Dipicolinic acid and 2-phosphonobutane-1,2,4-tricarboxylic acid can also be suitable. Glycinamide-N,N′-disuccinic acid (GADS), ethylenediamine-N-N′-diglutaric acid (EDDG) and 2-hydroxypropylenediamine-N-N′-disuccinic acid (HPDDS) can also be suitable.

The heavy metal ion sequestering agents may be used as their alkali or alkaline earth metal salts. Preferred chelants are tetrasodium EDTA and DPTA.

The anti-oxidant (including chelants if used) should be selected such that it slows the rate of the reaction between the particular developer and coupler compounds which are present, in the conditions under which the reaction takes place, and thus acts as an antioxidant.

In preferred processes the antioxidant is not provided wholly by one or more chelants.

Normally antioxidant is included in an amount of at least 0.01 and usually not more than 3 or 4, preferably not more than 2 wt %, based on total weight of composition applied to the hair. Suitable amounts of antioxidant include from 0.1 to 1.5 wt %, preferably not more than 1 wt %, especially not more than 0.6 or 0.5 wt %. Amounts of 0.4 to 0.5 wt % are often suitable. Amounts of above 0.5 or 0.6 wt % can also be advantageous, however.

We have found that the reaction between the defined developer and the defined couplers is potentially very fast and efficient. We believe that this rapid reaction, if not controlled, can lead to the majority of the developer and coupler molecules reacting before significant amounts have had an opportunity to diffuse into the hair shaft. Consequently the coloured molecules are formed on the outer surface of the hair shaft. These molecules are large and tend not to diffuse into the hair shaft. They are therefore highly susceptible to being washed out or otherwise removed. We believe that the inclusion of the antioxidant slows the otherwise very rapid reaction and allows diffusion of the developer and coupler molecules into the hair shaft before reaction, so that the coloured dimers are formed inside the hair shaft and are thus trapped.

We find this effect particularly surprising in view of the fact that it is known to supply components for hair dye in separate bottles, one of which contains oxidising agent and one of which contains a mixture of developers and couplers of the normal oxidative type, which contains small amounts of antioxidant for storage stability. However, the presence or absence of antioxidant makes no difference to wash fastness and fade resistance of the colours produced by standard oxidative dyes. It is only with the claimed combinations of developer and coupler that we find the antioxidant has the significant effect in improving wash fastness.

We have found that a further way of improving wash fastness is by selection of particular materials in the classes (A), (B) and (C) above. Such systems are described in our copending application number 9710759.3.

In these systems the couplers are selected from

(a) (1) naphthols having an active leaving group in the para-position relative to the OH group,

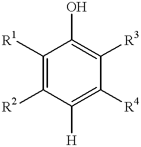

or (2) phenols of the formula

in which the H para to the OH group is an active leaving group and R¹, R², R³ and R⁴ are each independently selected from the group consisting of H, OH, methyl, ethyl, n-propyl, i-propyl, t-butyl, NH₂, CO₂H, CO₂R and COR, in which R is substituted or unsubstituted alkyl or alkenyl,

(b) 1,3-diketones (1) containing the group

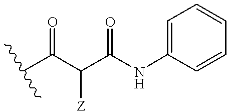

in which the N-phenyl group has no carboxy substituents, or (2) containing the group

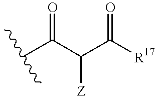

in which R¹⁷ contains an aryl group, or (3) of the formula

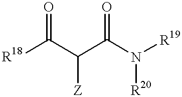

in which R¹⁸ is selected from the group consisting of methyl, ethyl, n-propyl, i-propyl, t-butyl and phenyl and R¹⁹ and R²⁰ are each independently selected from the group consisting of methyl, ethyl, n-propyl, hydroxymethyl and hydroxypropyl, or (4) of the formula

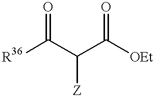

in which R³⁶ is C₄ alkyl,

in all of which Z is an active leaving group, and

(c) compounds of the formula

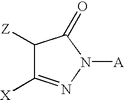

in which X is a non-leaving substituent and in which Z is an active leaving group and in which A is H or methyl,

and such that in the presence of an oxidising agent the or each developer reacts with the or each coupler substantially only at the position having the active leaving group

and provided that the composition includes at least one coupler (b) and/or at least one coupler (c).

Thus the compositions of this embodiment of the invention include at least one developer (i) and at least one coupler (ii). The coupler (ii) includes at least one coupler (b) and/or at least one coupler (c), i.e. one or more couplers (b) or one or more couplers (c) or at least one of each. Coupler (b) can be selected from couplers (b)(1), (b)(2) and (b)(3). The composition may also contain at least one coupler (a), which can be selected from couplers (a)(1) and (a)(2).

Further, certain of the coloured dimers formed have significantly reduced water-solubility in comparison with the trimers formed in standard oxidative colouring systems, which assists in achieving increased wash fastness and fade resistance in comparison with standard oxidative colouring systems. In some cases the structure of the developers and couplers is such that they show a greater degree of diffusion into the hair shaft than the colouring agents in known systems, and consequently lead to improved fade resistance.

The couplers (a) give a cyan colour, since they are a subset of couplers (A) discussed above. The particular shade or intensity of colour can be varied by varying the substituents of the phenol molecule (in cases (a)(2)) or naphthol molecule (in cases (a)(1)). It has an active leaving group in the para-position relative to the OH group. In case (2) this is an active proton, ie the aromatic ring is unsubstituted in the para-position and other substituents on the ring are not such as to reduce the reactivity at this position. In case (1) it may also be an active proton, or another active leaving group.

Couplers (a) may be (1) naphthols having an active leaving group in the para-position relative to the OH group. In this case they generally have the formula VIII, as follows:

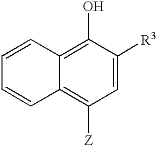

in which R³ is H, OH, —CO₂H, —CO₂R, F, Cl, Br, —CN, —NO₂, —CF₃, cycloalkyl, alkenyl, cycloalkenyl, aryl, alkaryl, aralkyl, —NH₂, —NHR, —NHCOR, —NR₂, —NHCOR, —R′NHCOR, —CONHR, R′CONHR, —R′OH, —SO₂R, SO₂NHR, —R′SO₂R, —R′SO₂NHR, —SO₃H, —OR, —R′OR or —COR, in any of which R is H, alkyl, cycloalkyl, alkenyl, cycloalkenyl, aryl, alkaryl or aralkyl, and R′ is alkylene, alkenylene, cycloalkylene, cycloalkenylene, arylene, alkarylene or aralkylene, or substituted versions of any of these. R³ may for instance be alkyl, cycloalkyl ,alkenyl, cycloalkenyl, aryl, alkaryl, aralkyl, R′NHCOR, R′CONHR, SO₂R, SO₂NHR, R′SO₂R or R′SO₂NHR. R³ may include an aryl group. It is preferred that R³ is H.

Preferably R³ is H. In other suitable couplers (a) R³ is

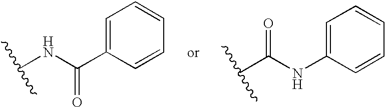

Suitable couplers (a)(1) thus have the formula XIV or XV, as follows:

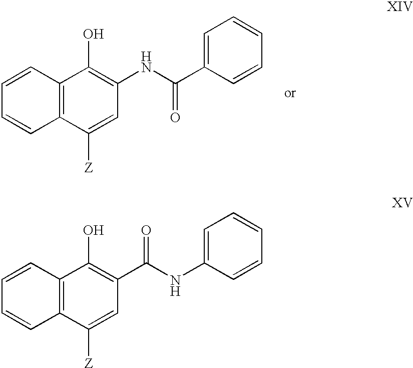

When coupler (a) is a naphthol (1) it is preferred that it has no solubilising substituents (other than Z), in particular no —COOH or —OH substituents. Coupler (a) (1) is preferably an unsubstituted naphthol.

We find that couplers of the formula XIII, in particular when R³ is H. and especially when Z is H, have an advantageous combination of properties for improving wash fastness whilst allowing rapid colouring. We believe this is because their molecules have a structure such that as monomers they are small enough to diffuse into the hair shaft but as dimers they are trapped within the hair shaft. Further, the solubility of the dimers is such that they are not easily washed out during subsequent hair treatment processes. They are particularly advantageous for colouring damaged hair (eg. hair which has been previously coloured, permed and/or bleached).

In particular these cyan couplers (a) (1) of the invention show improved fade resistance properties, especially on damaged hair, in comparison with those of GB 1,025,916.

Alternatively, the couplers (a) can be selected from a specific defined group of phenols (2) having the formula XVI

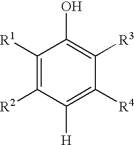

in which the H para to the OH group is an active leaving group and R¹, R², R³ and R⁴ are each independently selected from the group consisting of H, OH, methyl, ethyl, n-propyl, i-propyl, t-butyl, NH₂, CO₂H, CO₂R and COR, in which R is substituted or unsubstituted alkyl or alkenyl.

In this case the active leaving group is always H. Preferred couplers of this type include 3-aminophenol.

These preferred couplers (a) (2) also show particularly good performance on damaged hair. They demonstrate good colour uptake and good wash fastness. They also show good colour uptake and wash fastness on undamaged hair.

Yellow couplers (b) are 1,3-diketones. They may have one of two formulae. A first set (1) contain the group

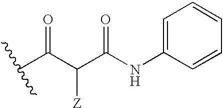

and generally have the formula XVII:

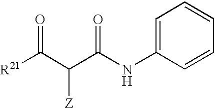

In these couplers the N-phenyl group may contain any non-interfering substituent, that is any group which does not hinder the colouring reaction between developer and coupler, with the exception that it may not contain any carboxy substituents. We find that molecules of this general type but having carboxy substituents, for instance those described in GB 1,025,916, exhibit reduced wash fastness in comparison with the couplers of the above structure.

Preferably also the N-phenyl contains no hydroxy substituents, and particularly preferably it contains no solubilising substituents. Most preferably it is unsubstituted.

The group R²¹ may be H, alkyl, cycloalkyl, alkenyl, cycloalkenyl, aryl, alkaryl, aralkyl, —R′NHCOR, —R′CONHR, —ROH, —R′SO₂R, —R′CO₂NHR, —NHCOR, —NR₂, —NHR, —NH₂, —R′OR or —OR. In these groups R can be H, alkyl, cycloalkyl, alkenyl, cycloalkenyl, aryl, alkaryl or aralkyl and R′ is alkylene, cycloalkylene, alkenylene, cycloalkenylene, arylene, alkarylene or aralkylene. Substituted versions of any of these can be used. Suitable substituents include OH, —OR, Cl, Br, F, —CO₂H, —CO₂R, —NH₂ and —COR. R may for instance be alkyl, cycloalkyl, alkenyl, cycloalkenyl, aryl, alkaryl, aralkyl, R′NHCOR, R′CONHR, SO₂R, SO₂NHR, R′SO₂R or R′SO₂NHR. Preferably R⁹ is alkyl, alkenyl, alkaryl, alkenaryl, aralkyl or aralkenyl. More preferably R²¹ is alkyl, aralkyl or alkaryl. Particularly preferred R²¹ groups are phenyl and C₁₋₃ alkyl, in particular ethyl and, especially, methyl.

Yellow couplers (b)(2) contain the group

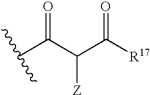

In particular they may have the following formula XVIII

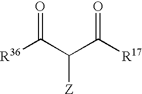

in which R¹⁷ contains an aryl group. Preferably R¹⁷ contains a phenyl group. It is preferred that the aryl, preferably phenyl, group does not contain any carboxy substituents. More preferably it does not contain any hydroxy substituents and particularly preferably it contains no solubilising substituents. In particular, R¹⁷ contains an unsubstituted phenyl group.

R³⁶ may for instance be alkyl, cycloalkyl, alkenyl, cycloalkenyl, aryl, alkaryl, aralkyl, —R′NHCOR, —R′CONHR, —ROH, —R′SO₂R, —R′CO₂NHR, —NHCOR, —NR₂, —NHR, —NH₂, —R′OR or —OR. It is preferably methyl.

A preferred group R¹⁷ is

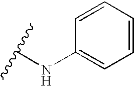

We find that couplers of this formula also have a particularly advantageous combination of properties for improving wash fastness whilst allowing rapid colouring, especially of damaged hair. In particular, they show improved wash fastness over the couplers of GB 1,025,916. We believe this is in part because of the presence of the aryl group in group R¹⁷, which increases the size of the final dimer trapped within the hair shaft.

Further preferred yellow couplers (b) (3) have the formula XIX

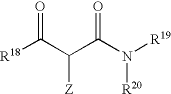

in which R¹⁸ is selected from the group consisting of methyl, ethyl, n-propyl, i-propyl, t-butyl and phenyl and R¹⁹ and R²⁰ are independently selected from the group consisting of methyl, ethyl, n-propyl, hydroxymethyl and hydroxypropyl.

Couplers (b) (3) are particularly advantageous for the coloration of undamaged as well as damaged hair. On undamaged hair they show fast colour uptake without loss of wash fastness. They also show good wash fastness and fade resistance on damaged hair.

A further coupler (b)(4) is of the formula XXX

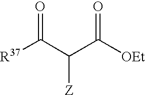

in which R³⁷ is C₄ alkyl, preferably t-butyl.

Magenta couplers (c) have the formula VIII

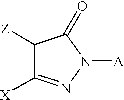

in which X is a non-leaving substituent and in which Z is an active leaving group and in which A is H or methyl.

Preferred couplers (c) have the formula XXI

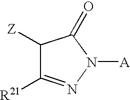

in which R²¹ is selected from the group consisting of H, methyl, ethyl, n-propyl, i-propyl, t-butyl and phenyl.

Couplers of the formulae XX and XXI are particularly suitable for colouring undamaged hair and give good fade resistance and colour uptake on undamaged as well as damaged hair.

In the above couplers (a), (b) and (c) the listed groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂, NHCH₃, SO₃H, C₁₋₃ alkyl such as ethyl or propyl and CONHR in which R is preferably C₁₋₃ alkyl. Phenyl groups in particular may contain one or more substituents which are the same or different. If phenyl groups are substituted, mono substitution is preferred. Preferably groups are unsubstituted unless otherwise stated.

In all the above couplers (a), (b) and (c), Z may be any of the active leaving groups listed for couplers (A), (B) and (C) above.

Specific examples of couplers (a) include α-naphthol, 3-aminophenol and the compounds having the following structural formulae:

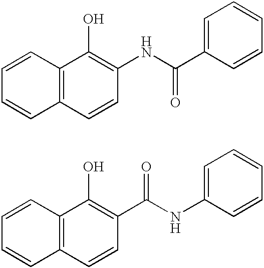

Specific examples of couplers (b) include benzoylacetanilide, acetoacetanilide, N,N-diethyl and N,N-dimethyl acetoacetamide and the compounds of the formulae

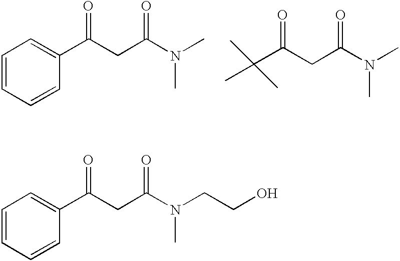

Specific examples of couplers (c) include the pyrazolone of structural formula:

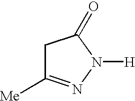

and compounds having the same formula except that Me is replaced by H or phenyl and/or H is replaced by Me.

We find that the use of these particular defined couplers (a), (b) and (c) gives particularly effective wash fastness and fade resistance. The couplers (a), in particular (a) (1) are particularly useful for obtaining high fade resistance on damaged hair. Couplers (a) (2) are especially suited to obtaining high fade resistance on undamaged hair.

Yellow couplers (b) are particularly useful for colouring damaged hair. Couplers (b)(3) are especially useful for colouring undamaged hair.

Magenta couples (c) are especially suitable for obtaining high fade resistance on undamaged hair.

The composition of the invention may contain any one, or more, of the couplers (A), (B) and (C) in combination with the defined developer and the antioxidant (if used). A particular advantage of the use of these particular couplers is that it is possible to obtain the full range of colours using just three specific types of coupler and one type of developer. Preferably the composition contains at least two out of the three types of coupler. In particular it contains at least one coupler (B) or (C). More preferably it contains at least one coupler of each type (A), (B) and (C). In some preferred compositions not more than two or even only one compound of any or all of the types (A), (B) and (C) is included.

This allows the couplers to be supplied in the form of a mixture having the required amounts of each type of coupler to develop whichever colour is desired. Alternatively, it also allows supply of the coupler materials separately packaged so that the consumer can control the eventual colour which is obtained by mixing the correct amounts of each coupler to form the composition.

When the composition contains the preferred cyan couplers (a), the preferred yellow couplers (b) and/or the preferred magenta couplers (c), it may contain at least one of each of these types of coupler. Preferably it contains at least two out of the three types and in particular preferably contains at least one coupler (b) and/or at least one coupler (c). It may in addition include further couplers of the types (A), (B) and (C), which are not within the definitions (a), (b) or (c).

Usually coupler is present in the composition in a total amount of from 0.001% to 5 or 10% by weight based on total weight of composition applied to the hair. Preferably total amounts of coupler are at least 0.01 wt %, often at least 0.1 or 1%. Preferably they are not more than 6%, and can in some preferred compositions be present in amounts up to 3%, for instance not more than 2.5%.

Couplers of types (A) and (C) can be used in particularly low amounts. For instance couplers of type (A) may be used in amounts, by weight based on total weight of composition applied to the hair, of from 0.001 to 1%, preferably 0.004 or 0.005 to 0.5%, for instance not more than 0.05 wt %. Couplers of type (C) can be used in amounts of from for instance 0.01 to 2 or 4%, preferably 0.03 to 3 or 2%, and in some compositions not more than 1 or 0.5%. Couplers of type (B) are often used in larger amounts, for instance from 0.05 to 3 or 4wt % (but in some cases up to 5 or 6%), for instance 0.1 to 2 or 3wt %. The specific preferred couplers (a), (b) and (c) may be included in these amounts. If couplers (a), (b) and (c) and additional couplers (A), (B) and (C) are used, preferably the total amounts of each type of coupler fall within these ranges.

Developer is often included in the composition in amounts of from 0.01 to 5 or 7% by weight based on total composition applied to the hair. Preferred amounts of developer are from 0.3 to 2 or 4%, preferably 0.4 to 1.5 or 3%.

For both developers and couplers the solubility properties can be important. The developer and coupler compounds themselves should have solubility such that they can be formulated in appropriate concentrations. For application at high pH they preferably have solubility of at least 10 g, more preferably at least 15g and most preferably at least 20 g/100 ml deionised water at pH about 10 and 25° C. They may have solubility at least 25 g/100 ml, and even up to 50 or 80 g/100 ml but normally not more than 30 g/100 ml.

The developer and coupler compounds are also generally such that the solubility of the final coloured dimer (or trimer if produced) is low under normal hair conditions and, especially, conditions of washing. Thus solubility (at pH about 8) of the final coloured molecule is preferably below 5 g/100 ml deionised water at 25° C., in particular below 2 or 1 g/100 ml and most preferably below 0.5 g/100 ml or even below 0.2 g/100 ml.

We find that if the developer and coupler compounds are sufficiently soluble in the composition, under the conditions of application to the hair, they will diffuse sufficiently rapidly into the hair shaft. However, the coloured molecules produced should be of sufficiently low solubility that they resist washing out of the hair. In compositions which are to be applied at high pH (for instance above pH 10), an indication of solubility can sometimes be given by pKa. Thus if one or more of the developers and couplers, in particular the developers, has an ionisable group which is substantially ionised at a pH of above 9, preferably above pH 10 this is an indicator of solubility at about pH 10. However, in the final coloured molecule and at the pH in the hair shaft (which is usually about pH 5.5 to 6) it becomes non-ionised. This gives an indication that under normal conditions it has reduced solubility. This can often be achieved by providing at least one group which has a pKa of from 8 to 12 (and is thus ionised above that pH) in a developer or coupler molecule and which on reaction to form a final coloured molecule also has pKa of from 8 to 12 (and is thus non-ionised at below that pH). Solubility can be affected by various factors but pKa can be a good indicator of likely solubility in some cases.

We find that an advantage of the colouring compounds of the invention is that they can give even coloration and fade resistance on both damaged and undamaged hair. This is particularly useful in cases where the hair has been dyed once and then allowed to grow so that undyed, undamaged hair appears. On redyeing, the undamaged hair and the faded, dyed, damaged hair must both be coloured and show even fade resistance. It is particularly important to be able to provide colour, wash fastness and fade resistance to damaged (eg bleached and/or permed and/or previously dyed) hair.

An advantage of the composition of the invention is that the full range of colours can be achieved using a very small number of compounds, in contrast to standard oxidative dyeing systems. Preferably only one or two, in particular only one, developer compound is used. In particular it is preferred that this is used in combination with not more than three, preferably only one or only two, compounds of any of the types (A), (B) and (C).

All of these couplers and developers can be classed as “oxidative” colouring agents, since they require the presence of an oxidising agent to initiate their reaction. Preferably these are the only oxidative colouring agents present in the composition and less than 0.1 wt %, in particular less than 0.05 or 0.08 wt %, and especially substantially no oxidative colouring agents are included which are not of the types (i) and (A), (B) and (C), preferably the formulae discussed above.

It is preferred that the composition contains less than 0.1 wt %, especially less than 0.08 wt % and in particular less than 0.05 wt % and even substantially no oxidative dye materials which are capable of undergoing reaction more than once (under the oxidising conditions of the hair colouring reaction).

A preferred composition comprises not more than 0.1 wt % of any oxidative colouring agent which can react with itself under the conditions of hair colouring. Preferably it comprises not more than 0.08 wt % or 0.05 wt % of any such agent. More preferably the total amount of such agents does not exceed these values.

Other colouring agents such as vegetable dyes can be included, but it is preferred that no non-oxidative dyes are present and indeed preferably no other colouring components are included than the developer (i) as defined and couplers (A), (B) and (C). That is, in the hair dye composition the colouring components consist essentially of developer (i) and couplers (A), (B) and/or (C). Trivial amounts of other colouring components can of course be included provided they do not significantly influence the final colour.

For the developers and couplers to be effective in forming colour they require the presence of an oxidising agent. This oxidising agent is normally included in the composition just before it is applied to the hair. Normally the composition of the invention will be supplied in at least two individual packages such as bottles, the oxidising agent being included in one package and the developers and couplers being included in another.

A preferred oxidising agent is hydrogen peroxide. Other oxidising agents which may be used include other inorganic peroxygen oxidising agents, preformed organic peroxyacid oxidising agents and other organic peroxides such as urea peroxide, melamine peroxide, and mixtures of any of these.

Suitable oxidising agents are preferably water-soluble, that is they have a solubility of at least about log in 1,000 ml of deionised water at 25° C. (“Chemistry” C. E. Mortimer, 5th Edition, page 277).

Suitable inorganic alkali metal peroxides other than hydrogen peroxide include sodium periodate, sodium perbromate and sodium peroxide, and inorganic perhydrate salt oxidising compounds such as the alkali metal salts of perborates, percarbonates, perphosphates, persilicates, and persulphates. Inorganic perhydrate salts may be incorporated as monohydrates, tetrahydrates etc. Mixtures of two or more of such inorganic peroxygen oxidising agents can be used if desired. Alkali metal bromates and iodates are suitable, bromates being preferred.

Another suitable inorganic oxidising agent is chlorite.

Amounts of inorganic peroxygen oxidising agent which can be used in the composition are normally from 0.0003 mol 0.2 mol per long of composition, preferably up to 0.1 mol/100 g.

Suitable preformed organic peroxyacid oxidising agents have the general formula R²³C(O)OOH, in which R²³ is selected from saturated or unsaturated, substituted or unsubstituted, straight or branched chain, alkyl, aryl or alkaryl groups with from 1 to 14 carbon atoms.

One class of organic peroxyacid compounds suitable for use in the invention is that of the amide substituted compounds of the following general formulae XXII and XXIII:

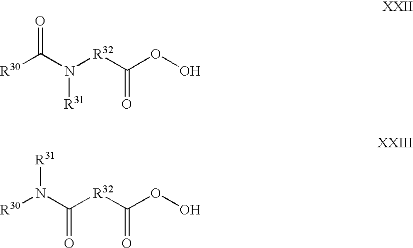

wherein R³⁰ is a saturated or unsaturated alkyl or alkaryl group or an aryl group, having from 1 to 14 carbon atoms, R⁼is a saturated or unsaturated alkyl or alkaryl group, or an aryl group, having from 1 to 14 carbon atoms and R³¹ is H or a saturated or an unsaturated alkyl or alkaryl group, or an aryl group, having from 1 to 10 carbon atoms. Amide substituted organic peroxyacid compounds of this type are described in EP-A-170,386.

Other suitable organic peroxyacid oxidising agents include peracetic, pernanoic, nonylamidoperoxycaproic acid (NAPCA), perbenzoic, m-chloroperbenzoic, di-peroxy-isophthalic, mono-peroxyphthalic, peroxylauric, hexanesulphonyl peroxy propionic, N,N-phthaloylamino peroxycaproic, monoper succinic, nonanoyloxybenzoic, dodecanedioyl-monoperoxybenzoic, nonylamide of peroxyadipic acid, diacyl and tetraacylperoxides, especially diperoxydodecanedioic acid, diperoxytetradecanedioic acid and diperoxyhexadecanedioic acid and derivatives thereof. Mono- and diperazelaic acid, mono- and diperbrassylic acid and N-phthaloylaminoperoxicaproic acid and derivatives thereof are also suitable for use in the invention.

Preferred peroxyacid materials are selected from peracetic and pernanoic acids and mixtures thereof. Suitable amounts of preformed organic peroxyacid oxidising agents are from about 0.0001 to 0.1 mol per 100 g of compositions, preferably from about 0.001 to 0.05 mol, more preferably from about 0.003 to 0.04 mol, especially from about 0.004 to 0.03 mol/100 g.

The preformed organic peroxyacid oxidising agent, where present, is preferably present at a level of from about 0.01% to about 8%, more preferably from about 0.1% to about 6%, most preferably from about 0.2% to about 4%, and especially from about 0.3% to about 3% by weight of the hair colouring composition. The weight ratio of the inorganic peroxygen oxidising agent to the preformed organic peroxy acid is preferably in the range of from about 0.0125:1 to about 500:1, more preferably from about 0.0125:1 to about 50:1.

If additional organic peroxides are used, suitable amounts are from about 0.01% to about 3%, preferably from about 0.01% to about 2%, more preferably from about 0.1% to about 1.5% and most preferably from about 0.2% to about 1% by weight of composition.

An advantage of the systems of the invention is that very low levels of oxidising agent can be used if desired. Such systems are described in more detail in our copending application number 9710756.9 filed today.

Usually the colouring compositions of the invention have pH above 6.1 or 6.5, often above pH 7, in particular above pH 8 or 9. A pH of from 9 to 12 is often suitable. The systems of the invention can also be incorporated into low pH (eg pH 1 to 6) hair colouring systems described in our copending application number GB9626713.3.

The composition may comprise ammonia, for instance in an amount of at least 0.01 wt %, preferably at least 0.05 wt % or 0.1 wt %.

The developers, couplers, antioxidant (if used) and oxidising agent, and any other materials to be applied to the hair as components of the composition of the invention, may be provided in any suitable physical form. A preferred physical form is liquid. The liquid may be of low viscosity, for instance it may be water-thin, or it may be of higher viscosity. The material may be suspended in a gel network. The gel may be solid or of low viscosity.

The materials for colouring the hair are often formulated so that when they are mixed to form the composition of the invention for application to the hair they form a product of cream-like consistency, which is convenient for application to the hair. The final composition which is applied to the hair is often in the form of an emulsion.

Each individual material may be supplied in a form such that the composition containing it has a pH of above or below 7. For instance it may be from pH 1 to 11. In order to assist solubility of the various components, particularly developers and couplers, in a water-based carrier, the carrier may have a pH of above 6.1 or 6.5 or even above 7, for instance from pH 8 or 9 to pH 10 or 11. A pH as supplied of from 1 to 6 can assist in improving stability of the components.

The materials may be provided such that the pH of the final composition when mixed for application to the hair has a pH below 7 even though one of the components used to form it has a pH of above 7. Alcohols such as ethanol in amounts of from for instance 5 to 10 or 25% may be included to aid solubility of the developers and, particularly, the couplers in a water-based carrier.

The composition often has a pH of at least 6.1, inparticular at least 6.5 or at least 7, for instance from 8 to 12.

In a second aspect of the invention we provide a method of colouring hair by providing

(i) one or more developers as defined above, and

(ii) one or more couplers as defined above, and

(iii) an oxidising agent, and

applying the components (i), (ii) and (iii) to the hair, preferably substantially simultaneously,

wherein the composition is such that when it is applied to hair by the hair switch colouring method described herein and washed 20 times by the washing protocol described herein, gives a value of ΔE fade, measured as described herein, as follows:

(a) when the composition is suitable for delivering a blonde or light brown shade ΔE fade is not more than 2.5

(b) when the composition is suitable for delivering a red shade, ΔE fade is not more than 5.0

(c) when the composition is suitable for delivering a black or dark brown shade ΔE fade is not more than 2.5.

In the method of the invention the components (i), (ii) and (iii) are preferably mixed to form a single composition and then applied to the hair together. However, within the term “substantially simultaneously” we also include application of one or more components to the hair followed by subsequent application of the remaining components within a period of not more than 5 minutes.

Application of the components substantially simultaneously, in particular mixing and application together, is especially beneficial to the consumer because of the increased convenience over sequential application.

Generally in the method of the invention the fade resistance ΔE fade obtained on the hair which is coloured using the method is as given under (a), (b) or (c) above.

The conditions of the reaction are normally those conventionally applied for dyeing hair. The temperature is normally from 10 to 45° C., often 20 to 35° C. pH can be low (eg below 7 or 6) but is often high, for instance above 6.5 or 7, or above 8 or 9 or even above 10.

In this specification, when leaving groups are discussed, as well as compounds which react only at one position or only with certain other compounds, we mean reaction in the conditions under which the colouring compounds will be applied to the hair.

The hair which is coloured may be damaged or undamaged or may be partially damaged and partially undamaged. Damage can be caused for instance by bleaching, perming or previous colouring.

In the method of the invention any of the additional features discussed above in connection with the compositions of the invention may be used.

Any of the compositions can contain various optional ingredients as follows.

Oxidative Dye Precursors

Preferably the only oxidative dye materials in the composition are materials (i) and (ii) discussed above. However, the compositions may optionally contain minor amounts of other oxidative dye materials. These may include those described in our copending application PCT/US97/22719, filed Dec. 9, 1997.

In general terms, oxidative dye primary intermediates include those monomeric materials which, on oxidation, form oligomers or polymers having extended conjugated systems of electrons in their molecular structure. Because of the new electronic structure, the resultant oligomers and polymers exhibit a shift in their electronic spectra to the visible range and appear coloured. For example, oxidative primary intermediates capable of forming coloured polymers include materials such as aniline, which has a single functional group and which, on oxidation, forms a series of conjugated imines and quinoid dimers, trimers, etc. ranging in colour from green to black. Compounds such as p-phenylenediamine, which has two functional groups, are capable of oxidative polymerization to yield higher molecular weight coloured materials having extended conjugated electron systems. A representative list of primary intermediates and secondary couplers suitable for use herein is found in Sagarin, “Cosmetic Science and Technology”,” Interscience, Special Ed. Vol. 2 pages 308 to 310.

Non-oxidative and Other Dyes

The hair colouring compositions used in the present invention may, in addition to the essential oxidative hair colouring agents (i) and (ii) and optional oxidative dyes, optionally include non-oxidative and other dye materials. Optional non-oxidative and other dyes suitable for use in the hair colouring compositions and processes according to the present invention include both semi-permanent, temporary and other dyes. Non-oxidative dyes as defined herein include the so-called “direct action dyes”, metallic dyes, metal chelate dyes, fibre reactive dyes and other synthetic and natural dyes. Various types of non-oxidative dyes are detailed in: “Chemical and Physical Behaviour of Human Hair” 3rd Ed. by Clarence Robbins (pp250-259); “The Chemistry and Manufacture of Cosmetics”. Volume IV. 2nd Ed. Maison G. De Navarre at chapter 45 by G. S. Kass (pp841-920); “Cosmetics: Science and Technology”, 2nd Ed., Vol. II, Balsam Sagarin, Chapter 23 by F. E. Wall (pp 279-343); “The Science of Hair Care” edited by C. Zviak, Chapter 7 pp 235-261 and “Hair Dyes”, J. C. Johnson, Noyes Data Corp., Park Ridge, U.S.A. (1973), (pp 3-91 and 113-139).

Direct action dyes, which do not require an oxidative effect in order to develop the color, are also designated hair tints and have long been known in the art. They are usually applied to the hair in a base matrix which includes surfactant material. Direct action dyes include nitro dyes such as the derivatives of nitroamino benzene or nitroaminophenol; disperse dyes such as nitroaryl amines, aminoanthraquinones or azo dyes; anthraquinone dyes, naphthoquinone dyes; basic dyes such as Acridine Orange C.I. 46005.

Nitro dyes are added to dyeing compositions to enhance colour of colorant and to add suitable aesthetic colour to the dye mixture prior to application.

Further examples of direct action dyes include the Arianor dyes basic brown 17, C.I.(color index)-no. 12,251; basic red 76, C.I.-12,245; basic brown 16, C.I.-12,250; basic yellow 57, C.I.-12,719 and basic blue 99, C.I.-56,059 and further direct action dyes such as acid yellow 1, C.I.-10,316 (D&C yellow no.7); acid yellow 9, C.I.-13,015; basic violet C.I.-45,170; disperse yellow 3, C.I.-11,855; basic yellow 57, C.I.-12,719; disperse yellow 1, C.I.-10,345; basic violet 1, C.I.-42,535, basic violet 3, C.I.-42,555; greenish blue, C.I.-42090 (FD&C Blue no.1); yellowish red, C.I.-14700 (FD&C red no.4); yellow, C.I.19140 (FD&C yellow no5); yellowish orange, C.I.15985 (FD&C yellow no.6); bluish green, C.I.42053 (FD&C green no.3); yellowish red, C.I.16035 (FD&C red no.40); bluish green, C.I.61570 (D&C green no.3); orange, C.I.45370 (D&C orange no.5); red, C.I.15850 (D&C red no.6); bluish red, C.I.15850 (D&C red no.7); slight bluish red, C.I.45380 (D&C red no.22); bluish red, C.I.45410 (D&C red no.28); bluish red, C.I.73360 (D&C red no.30); reddish purple, C.I.17200 (D&C red no.33); dirty blue red, C.I.15880 (D&C red no.34); bright yellow red, C.I.12085 (D&C red no.36); bright orange, C.I.15510 (D&C orange no.4); greenish yellow, C.I.47005 (D&C yellow no.10); bluish green, C.I.59040 (D&C green no.8); bluish violet, C.I.60730 (Ext. D&C violet no.2); greenish yellow, C.I.10316 (Ext. D&C yellow no.7);

Fibre reactive dyes include the Procion (RTM), Drimarene (RTM), Cibacron (RTM), Levafix (RTM) and Remazol (RTM) dyes available from ICI, Sandoz, Ciba-Geigy, Bayer and Hoechst respectively.

Natural dyes and vegetable dyes as defined herein include henna (Lawsonia alba), camomile (Matricaria chamomila or Anthemis nobilis), indigo, logwood and walnut hull extract.

Temporary hair dyes, or hair coloring rinses, are generally comprised of dye molecules which are too large to diffuse into the hair shaft and which act on the exterior of the hair. They are usually applied via a leave-in procedure in which the dye solution is allowed to dry on the hair surface. As such these dyes are typically less resistant to the effects of washing and cleaning the hair with surface active agents and are washed off the hair with relative ease. Temporary hair dye can be used in the compositions of the invention and examples of preferred temporary hair dyes are illustrated below.

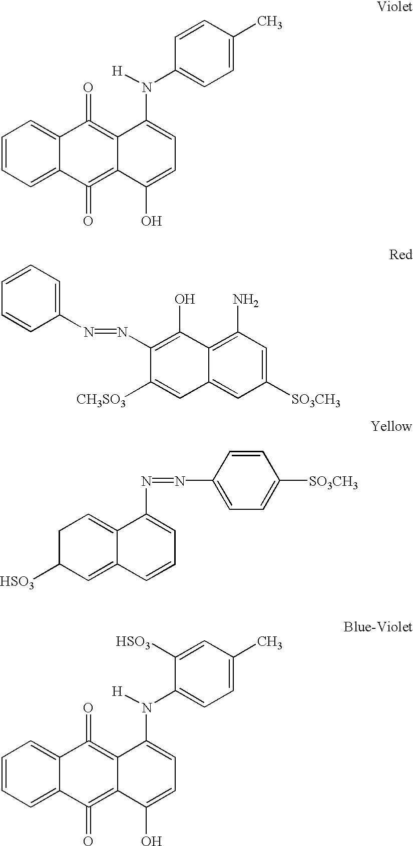

Semi-permanent hair dyes are dyes which are generally smaller in size and effect to temporary hair rinses but are generally larger than permanent (oxidative) dyes. Typically, semi-permanent dyes act in a similar manner to oxidative dyes in that they have the potential to diffuse into the hair shaft. However, semi-permanent dyes are generally smaller in size than the aforementioned conjugated oxidative dye molecules and as such are pre-disposed to gradual diffusion out of the hair again. Simple hair washing and cleaning action will encourage this process and in general semi-permanent dyes are largely washed out of the hair after about 5 to 8 washes. A semi-permanent dye system can be included in the compositions of the present invention. Suitable semi-permanent dyes for use in the compositions of the present invention are HC Blue 2, HC Yellow 4, HC Red 3, Disperse Violet 4, Disperse Black 9, HC Blue 7, HC Yellow 2, Disperse Blue 3, Disperse Violet 1 and mixtures thereof. Examples of semi-permanent dyes are illustrated below:

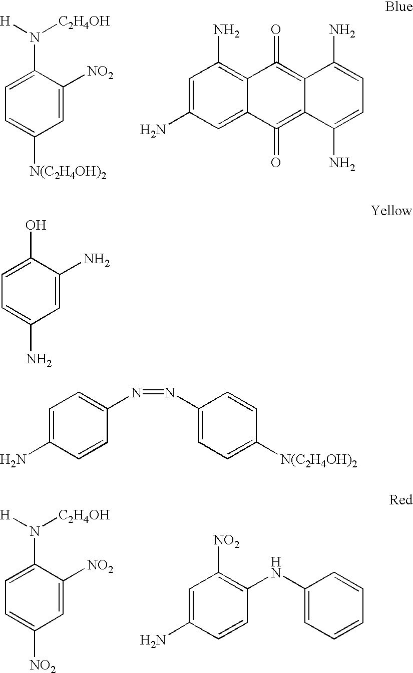

Typical semi-permanent dye systems incorporate mixtures of both large and small colour molecules. As the size of the hair is not uniform from root to tip the small molecules will diffuse both at the root and tip, but will not be retained within the tip, while the larger molecules will be generally only be able to diffuse into the ends of the hair. This combination of dye molecule size is used to help give consistent color results from the root to the tip of the hair both during the initial dyeing process and during subsequent washing.

Buffering Agents

If so desired, the compositions may contain one or more optional buffering agents and/or hair swelling agents (HSAs). Several different pH modifiers can be used to adjust the pH of the final composition or any constituent part thereof.

This pH adjustment can be effected by using well known acidifying agents in the field of treating keratinous fibres, and in particular human hair, such as inorganic and organic acids such as hydrochloric acid, tartaric acid, citric acid, succinic acid, phosphoric acid and carboxylic or sulphonic acids such as ascorbic acid, acetic acid, lactic acid, sulphuric acid, formic acid, ammonium sulphate and sodium dihydrogenphosphate/phosphoric acid, disodium hydrogenphosphate/phosphoric acid, potassium chloride/hydrochloric acid, potassium dihydrogen phthalate/hydrochloric acid, sodium citrate/hydrochloric acid, potassium dihydrogen citrate/hydrochloric acid, potassium dihydrogencitrate/citric acid, sodium citrate/citric acid, sodium tartarate/tartaric acid, sodium lactate/lactic acid, sodium acetate/acetic acid, disodium hydrogenphosphate/citric acid and sodium chloride/glycine/hydrochloric acid, succinic acid and mixtures thereof. These are suitable for buffering to low pH.

Examples of alkaline buffering agents are ammonium hydroxide, ethylamine, dipropylamine, triethylamine and alkanediamines such as 1,3-diaminopropane, anhydrous alkaline alkanolamines such as mono or di-ethanolamine, preferably those which are completely substituted on the amine group such as dimethylaminoethanol, polyalkylene polyamines such as diethylenetriamine or a heterocyclic amine such as morpholine as well as the hydroxides of alkali metals, such as sodium and potassium hydroxide, hydroxides of alkali earth metals, such as magnesium and calcium hydroxide, basic amino acids such as L-arginine, lysine, alanine, leucine, iso-leucine, oxylysine and histidine and alkanolamines such as dimethylaminoethanol and aminoalkylpropanediol and mixtures thereof. Also suitable for use herein are compounds that form HCO₃⁻ by dissociation in water (hereinafter referred to as “ion forming compounds”). Examples of suitable ion forming compounds are Na₂CO₃, NaHCO₃, K₂CO₃, (NH4)₂CO₃, NH₄HCO₃, CaCO₃ and Ca(HCO₃) and mixtures thereof. These are suitable for buffering to high pH.

Preferred for use herein as buffering agents (to low pH) are organic and inorganic acids having a first pKa below pH 6, and their conjugate bases. As defined herein, first pKa means the negative logarithm (to the base 10) of the equilibrium constant, K, where K is the acid dissociation constant. Suitable organic and inorganic acids for use herein are: aspartic, maleic, tartaric, glutamic, glycolic, acetic, succinic, salicylic, formic, benzoic, malic, lactic, malonic, oxalic, citric, phosphoric acid and mixtures thereof. Particularly preferred are acetic, succinic, salicylic and phosphoric acids and mixtures thereof.

Catalyst

The colouring compositions herein may optionally contain a catalyst for any inorganic peroxygen oxidising agents and the optional preformed peroxy acid oxidising agent(s).

Thickeners

The colouring compositions of the present invention may additionally include a thickener at a level of from about 0.05% to about 20%, preferably from about 0.1% to about 10%, more preferably from about 0.5% to about 5% by weight. Thickening agents suitable for use in the compositions herein are selected from oleic acid, cetyl alcohol, oleyl alcohol, sodium chloride, cetearyl alcohol, stearyl alcohol, synthetic thickeners such as Carbopol, Aculyn and Acrosyl and mixtures thereof. Preferred thickeners for use herein are Aculyn 22 (RTM,) steareth-20 methacrylate copolymer; Aculyn 44 (RTM) polyurethane resin and Acusol 830 (RTM), acrylate copolymers which are available from Rohm and Haas, Philadelphia, Pa., USA. Additional thickening agents suitable for use herein include sodium alginate or gum arabic, or cellulose derivatives, such as methyl cellulose or the sodium salt of carboxymethylcellulose or acrylic polymers.

Diluent

Water is the preferred diluent for the compositions according to the present invention. However, the compositions according to the present invention may include one or more solvents as additional diluent materials. Generally, solvents suitable for use in the coloring compositions of the present invention are selected to be miscible with water and innocuous to the skin. Solvents suitable for use as additional diluents herein include C₁-C₂₀ mono- or polyhydric alcohols and their ethers, glycerine, with monohydric and dihydric alcohols and their ethers preferred. In these compounds, alcoholic residues containing 2 to 10 carbon atoms are preferred. Thus, a preferred group includes ethanol, isopropanol, n-propanol, butanol, propylene glycol, ethylene glycol monoethyl ether, 1,2-hexanediol, butoxyethanol, benzyl alcohol, and mixtures thereof. Water is the preferred principal diluent in the compositions according to the present invention. Principal diluent, as defined herein, means, that the level of that diluent present is higher than the total level of any other diluents.

The diluent is present at a level preferably of from about 5% to about 99.98%, preferably from about 15% to about 99.5%, more preferably at least from about 30% to about 99%, and especially from about 50% to about 98% by weight of the compositions herein.

Enzyme

A further additional material useful in the hair coloring compositions according to the present invention is one or more enzymes.

Suitable enzymatic materials include the commercially available lipases, cutinases, amylases, neutral and alkaline proteases, esterases, cellulases, pectinases, lactases and peroxidases conventionally incorporated into detergent compositions. Suitable enzymes are discussed in U.S. Pat. No. 3,519,570 and 3,533,139.

Peroxidases are haemoproteins specific for peroxide, but using a wide range of substances as donors. Catalase which decomposes peroxide, is included here in view of the fact that it is generally similar in structure and properties and is able to bring about certain oxidations by H₂O₂. The decomposition of H₂O₂ can be regarded as the oxidation of one molecule by the other. It is widespread in aerobic cells and may have some more important function. The coenzyme peroxidases are not haemoproteins and one at least is a flavoprotein. Other flavoproteins such as xanthine oxidase will also use H₂O₂ among other acceptors, and the coenzyme peroxidases resemble these rather than the classical peroxidases in not being specific for H₂O₂. Suitable peroxidases for the compositions of the present invention include horseradish peroxidase, Japanese radish peroxidase, cow's milk peroxidase, rat liver peroxidase, linginase and haloperoxidase such as chloro- and bromo-peroxidase.

Enzymes are optionally incorporated at levels sufficient to provide up to about 50 mg by weight, more typically about 0.01 g to about 10 mg of active enzyme per gram of the hair treatment composition of the invention. Stated otherwise the peroxidase enzyme may be incorporated into the compositions in accordance with the invention at a level of from about 0.0001% to about 5%, preferably from about 0.001% to about 1%, more preferably from about 0.01% to about 1% active enzyme by weight of the composition.

Commercially available protease enzymes include those sold under the trade names Alcalase, Savinase, Primase, Durazym, and Esperase by Novo Industries A/S (Denmark), those sold under the tradename Maxatase, Maxacal and Maxapem by Gist-Brocades, those sold by Genencor International, and those sold under the tradename Opticlean and Optimase by Solvay Enzymes. Protease enzyme may be incorporated into the compositions in accordance with the invention at a level of from 0.0001% to 4% active enzyme by weight of the composition.

Amylases include, for example, α-amylases obtained from a special strain of B. licheniformis, described in more detail in GB-1,269,839 (Novo). Preferred commercially available amylases include for example, those sold under the tradename Rapidase by Gist-Brocades, and those sold under the tradename Termamyl and BAN by Novo Industries A/S. Amylase enzyme may be incorporated into the composition in accordance with the invention at a level of from 0.0001% to 2% active enzyme by weight of the composition.

Lipolytic enzyme may be present at levels of active lipolytic enzyme of from 0.0001% to 2% by weight, preferably 0.001% to 1% by weight, most preferably from 0.001% to 0.5% by weight of the compositions.

The lipase may be fungal or bacterial in origin being obtained, for example, from a lipase producing strain of Humicola sp., Thermomyces sp. or Pseudomonas sp. including Pseudomonas pseudoalcaligenes or Pseudomonas fluorescens. Lipase from chemically or genetically modified mutants of these strains are also useful herein. A preferred lipase is derived from Pseudomonas pseudoalcaligenes, which is described in Granted European Patent, EP-B-0218272.

Another preferred lipase herein is obtained by cloning the gene from Humicola lanuginosa and expressing the gene in Aspergillus oryza, as host, as described in European Patent Application, EP-A-0258 068, which is commercially available from Novo Industri A/S, Bagsvaerd, Denmark, under the trade name Lipolase. This lipase is also described in U.S. Pat. No. 4,810,414, Huge-Jensen et al, issued Mar. 7, 1989.

Surfactant Materials

The compositions of the present invention can additionally contain a surfactant system. Suitable surfactants for inclusion in the compositions of the invention generally have a lipophilic chain length of from about 8 to about 22 carbon atoms and can be selected from anionic, cationic, nonionic, amphoteric, zwitterionic surfactants and mixtures thereof.

(i) Anionic Surfactants

Anionic surfactants suitable for inclusion in the compositions of the invention include alkyl sulphates, ethoxylated alkyl sulphates, alkyl glyceryl ether sulfonates, methyl acyl taurates, fatty acyl glycinates, N-acyl glutamates, acyl isethionates, alkyl sulfosuccinates, alkyl ethoxysulphosuccinates, alpha-sulfonated fatty acids, their salts and/or their esters, alkyl ethoxy carboxylates, alkyl phosphate esters, ethoxylated alkyl phosphate esters, alkyl sulphates, acyl sarcosinates, hydrotropes, such as alkyl xylene sulphonate and fatty acid/protein condensates, and mixtures thereof. Alkyl and/or acyl chain lengths for these surfactants are C₁₂-C₂₂, preferably C₁₂-C₁₈, more preferably C₁₂-C₁₄.

(ii) Nonionic Surfactants

The compositions of the invention can also comprise water-soluble nonionic surfactant(s). Surfactants of this class include C₁₂-C₁₄ fatty acid mono- and diethanolamides, sucrose polyester surfactants and polyhydroxy fatty acid amide surfactants having the general formula below.

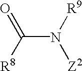

The preferred N-alkyl, N-alkoxy or N-aryloxy, polyhydroxy fatty acid amide surfactants according to the above formula are those in which R⁸is C₅-C₃₁ hydrocarbyl, preferably C6-C₁₉ hydrocarbyl, including straight-chain and branched chain alkyl and alkenyl, or mixtures thereof and R⁹is typically hydrogen, C₁-C₈ alkyl or hydroxyalkyl, preferably methyl, or a group of formula —R¹—O—R² wherein R¹ is C₂-C₈ hydrocarbyl including straight-chain, branched-chain and cyclic (including aryl), and is preferably C₂-C₄ alkylene, R² is C₁-C₈ straight-chain, branched-chain and cyclic hydrocarbyl including aryl and oxyhydrocarbyl, and is preferably C₁-C₄ alkyl, especially methyl, or phenyl. Z² is a polyhydroxyhydrocarbyl moiety having a linear hydrocarbyl chain with at least 2 hydroxyls (in the case of glyceraldehyde) or at least 3 hydroxyls (in the case of other reducing sugars) directly connected to the chain, or an alkoxylated derivative (preferably ethoxylated or propoxylated) thereof. Z² preferably will be derived from a reducing sugar in a reductive amination reaction, and most preferably Z² is a glycityl moiety. Suitable reducing sugars include glucose, fructose, maltose, lactose, galactose, mannose, and xylose, as well as glyceraldehyde. As raw materials, high dextrose corn syrup, high fructose corn syrup, and high maltose corn syrup can be utilised as well as the individual sugars listed above. These corn syrups may yield a mix of sugar components for Z². It should be understood that it is by no means intended to exclude other suitable raw materials. Z² preferably will be selected from the group consisting of —CH₂—(CHOH)ₙ—CH₂OH, —CH(CH₂OH)—(CHOH)ₙ₋₁—CH₂H, CH₂(CHOH)₂ (CHOR′)CHOH)—CH₂OH, where n is an integer from 1 to 5, inclusive, and R′ is H or a cyclic mono- or polysaccharide, and alkoxylated derivatives thereof. As noted, most preferred are glycityls wherein n is 4, particularly —CH₂—(CHOH)₄—CH₂OH.

The most preferred polyhydroxy fatty acid amide has the formula R⁸(CO)N(CH₃)CH₂(CHOH)₄CH₂OH wherein R⁸ is a C6-C19 straight chain alkyl or alkenyl group. In compounds of the above formula, ^{R8}—CO—N< can be, for example, cocoamide, stearamide, oleamide, lauramide, myristamide, capricamide, palmiamide, tallowamide, etc.

Suitable oil-derived nonionic surfactants for use herein include water soluble vegetable and animal-derived emollients such as triglycerides with a polyethyleneglycol chain inserted; ethoxylated mono- and diglycerides, polyethoxylated lanolins and ethoxylated butter derivatives. One preferred class of oil-derived nonionic surfactants for use herein have the general formula below:

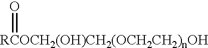

wherein n is from about 5 to about 200, preferably from about 20 to about 100, more preferably from about 30 to about 85, and wherein R comprises an aliphatic radical having on average from about 5 to 20 carbon atoms, preferably from about 7 to 18 carbon atoms.

Suitable ethoxylated oils and fats of this class include polyethyleneglycol derivatives of glyceryl cocoate, glyceryl caproate, glyceryl caprylate, glyceryl tallowate, glyceryl palmate, glyceryl stearate, glyceryl laurate, glyceryl oleate, glyceryl ricinoleate, and glyceryl fatty esters derived from triglycerides, such as palm oil, almond oil, and corn oil, preferably glyceryl tallowate and glyceryl cocoate.

Preferred for use herein are polyethyleneglycol based polyethoxylated C₉-C₁₅ fatty alcohol nonionic surfactants containing an average of from about 5 to about 50 ethyleneoxy moieties per mole of surfactant.

Suitable polyethylene glycol based polyethoxylated C₉-C₁₅ fatty alcohols suitable for use herein include C₉-C₁₁ Pareth-3, C₉-C₁₁, Pareth-4, C₉-C₁₁ Pareth-5, C₉-C₁₁, Pareth-6, C₉-C₁₁, Pareth-7, C₉-C₁₁, Pareth-8, C₁₁-C₁₅ Pareth-3, C₁₁-C₁₅ Pareth-4, C₁₁-C₁₅ Pareth-5, C₁₁-C₁₅ Pareth-6, C₁₁-C₁₅ Pareth-7, C₁₁-C₁₅ Pareth-8, C₁₁-C₁₅ Pareth-9, C₁₁-C₁₅ Pareth-10, C₁₁-C₁₅ Pareth-11, C₁₁-C₁₅ Pareth-12, C₁₁-C₁₅ Pareth-13 and C₁₁-C₁₅ Pareth-14. PEG 40 hydrogenated castor oil is commercially available under the tradename Cremophor (RTM) from BASF. PEG 7 glyceryl cocoate and PEG 20 glyceryl laurate are commercially available from Henkel under the tradenames Cetiol (RTM) HE and Lamacit (RTM) GML 20 respectively. C₉-C₁₁, Pareth-8 is commercially available from Shell Ltd under the tradename Dobanol (RTM) 91-8. Particularly preferred for use herein are polyethylene glycol ethers of ceteryl alcohol such as Ceteareth 25 which is available from BASF under the trade name Cremaphor A25.

Nonoxynol surfactants may also be used.

Also suitable for use herein are nonionic surfactants derived from composite vegetable fats extracted from the fruit of the Shea Tree (Butyrospermum Karkii Kotschy) and derivatives thereof. Similarly, ethoxylated derivatives of Mango, Cocoa and Illipe butter may be used in compositions according to the invention. Although these are classified as ethoxylated nonionic surfactants it is understood that a certain proportion may remain as non-ethoxylated vegetable oil or fat.

Other suitable oil-derived nonionic surfactants include ethoxylated derivatives of almond oil, peanut oil, rice bran oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, and sunflower seed oil.

(iii) Amphoteric Surfactants

Amphoteric surfactants suitable for use in the compositions of the invention include:

(a) imidazolinium surfactants of the formula (1)

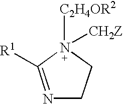

wherein R¹ is C₇-C₂₂ alkyl or alkenyl, R² is hydrogen or CH₂Z, each Z is independently CO₂M or CH₂CO₂M, and M is H, alkali metal, alkaline earth metal, ammonium or alkanolammonium; and/or ammonium derivatives of the formula (2)

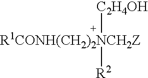

wherein R¹, R² and Z are as defined above;

(b) aminoalkanoates of the formula (3)

R¹NH(CH₂)ₙCO₂M

iminodialkanoates of the formula (4)

R¹N[(CH₂)ₘCO₂M]₂

and iminopolyalkanoates of the formula (5)

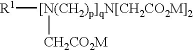

wherein n, m, p, and q are numbers from 1 to 4, and R¹ and M are independently selected from the groups specified above; and

(c) mixtures thereof.

Suitable amphoteric surfactants of type (a) are marketed under the trade name Miranol and Empigen and are understood to comprise a complex mixture of species. In practice, a complex mixture of cyclic and non-cyclic species is likely to exist and both definitions are given here for sake of completeness. Preferred for use herein, however, are the non-cyclic species.

Examples of suitable amphoteric surfactants of type (a) include compounds of formula (1) and/or (2) in which R₁ is C₈H₁₇ (especially iso-capryl), C₉H₁₉ and C₁₁H₂₃ alkyl. Especially preferred are the compounds in which R¹ is C₉H₁₉, Z is CO₂M and R² is H; the compounds in which R¹ is C₁₁H₂₃, Z is CO₂M and R² is CH₂CO₂M; and the compounds in which R¹ is C₁₁H₂₃, Z is CO₂M and R² is H.

In CTFA nomenclature, materials suitable for use in the present invention include cocoamphocarboxypropionate, cocoamphocarboxy propionic acid, and especially cocoamphoacetate and cocoamphodiacetate (otherwise referred to as cocoamphocarboxyglycinate). Specific commercial products include those sold under the trade names of Ampholak 7TX (sodium carboxy methyl tallow polypropyl amine), Empigen CDL60 and CDR 60 (Albright & Wilson), Miranol H2M Conc. Miranol C2M Conc. N.P., Miranol C2M Conc. O.P., Miranol C2M SF, Miranol CM Special (Rhône-Poulenc); Alkateric 2CIB (Alkaril Chemicals); Amphoterge W-2 (Lonza, Inc.); Monateric CDX-38, Monateric CSH-32 (Mona Industries); Rewoteric AM-2C (Rewo Chemical Group); and Schercotic MS-2 (Scher Chemicals). Further examples of amphoteric surfactants suitable for use herein include Octoxynol-1 (RTM), polyoxethylene (1) octylphenyl ether; Nonoxynol-4 (RTM), polyoxyethylene (4) nonylphenyl ether and Nonoxynol-9, polyoxyethylene (9) nonylphenyl ether.

It will be understood that a number of commercially-available amphoteric surfactants of this type are manufactured and sold in the form of electroneutral complexes with, for example, hydroxide counterions or with anionic sulfate or sulfonate surfactants, especially those of the sulfated C₈-C₁₈ alcohol, C₈-C₁₈ ethoxylated alcohol or C₈-C₁₈ acyl glyceride types. Note also that the concentrations and weight ratios of the amphoteric surfactants are based herein on the uncomplexed forms of the surfactants, any anionic surfactant counterions being considered as part of the overall anionic surfactant component content.

Examples of preferred amphoteric surfactants of type (b) include N-alkyl polytrimethylene poly-, carboxymethylamines sold under the trade names Ampholak X07 and Ampholak 7CX by Berol Nobel and also salts, especially the triethanolammonium salts and salts of N-lauryl-beta-amino propionic acid and N-lauryl-imino-dipropionic acid. Such materials are sold under the trade name Deriphat by Henkel and Mirataine by Rhône-Poulenc.

(iv) Zwitterionic Surfactants

Water-soluble auxiliary zwitterionic surfactants suitable for inclusion in the compositions of the present invention include alkyl betaines of the formula R⁵R⁶R⁷N⁺(CH₂)ₙCO₂M and amido betaines of the formula (6) below:

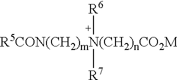

wherein R⁵ is C₁₁-C₂₂ alkyl or alkenyl, R⁶ and R⁷ are independently C₁-C₃ alkyl, N is H, alkali metal, alkaline earth metal, ammonium or alkanolammonium, and n, m are each numbers from 1 to 4. Preferred betaines include cocoamidopropyldimethylcarboxymethyl betaine, laurylamidopropyldimethylcarboxymethyl betaine and Tego betaine (RTM).

Water-soluble auxiliary sultaine surfactants suitable for inclusion in the compositions of the present invention include alkyl sultaines of the formula (7) below:

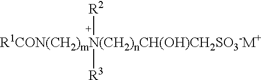

wherein R¹ is C₇ to C₂₂ alkyl or alkenyl, R² and R³ are independently C₁ to C₃ alkyl, M is H, alkali metal, alkaline earth metal, ammonium or alkanolammonium and m and n are numbers from 1 to 4. Preferred for use herein is coco amido propylhydroxy sultaine.

Water-soluble auxiliary amine oxide surfactants suitable for inclusion in the compositions of the present invention include alkyl amine oxide R⁵⁶R⁷NO and amido amine oxides of the formula (8) below:

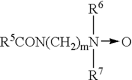

wherein R⁵ is C₁₁ to C₂₂ alkyl or alkenyl, R⁶ and R⁷ are independently C₁ to C₃ alkyl, M is H, alkali metal, alkaline earth metal, ammonium or alkanolammonium and m is a number from 1 to 4. Preferred amine oxides include cocoamidopropylamine oxide, lauryl dimethyl amine oxide and myristyl dimethyl amine oxide.

Additional Optional Materials

A number of additional optional materials can be added to the coloring compositions herein described, each at a level of from about 0.001% to about 5%, preferably from about 0.01% to about 3%, more preferably from about 0.05% to about 2% by weight of composition. Such materials include proteins and polypeptides and derivatives thereof; water-soluble or solubilizable preservatives; natural preservatives such as benzyl alcohol, potassium sorbate and bisabalol, benzoic acid, sodium benzoate and 2-phenoxyethanol; dye removers such as oxalic acid, sulphated castor oil, salicylic acid and sodium thiosulphate; H₂O₂ stabilisers; moisturising agents such as hyaluronic acid, chitin, and starch-grafted sodium polyacrylates such as Sanwet (RTM) IM-1000, IM-1500 and IM-2500 available from Celanese Superabsorbent Materials, Portsmith, Va., USA and described in U.S. Pat. No. 4,076,663 as well as methyl cellulose, starch, higher fatty alcohols, paraffin oils, fatty acids and the like; solvents; anti-bacterial agents such as Oxeco (phenoxy isopropanol); low temperature phase modifiers such as ammonium ion sources (e.g. NH₄ Cl); viscosity control agents such as magnesium sulfate and other electrolytes; quaternary amine compounds such as distearyl-, dilauryl-, di-hydrogenated beef tallow-, dimethyl ammonium chloride, dicetyldiethyl ammoniumethylsulphate, ditallowdimethyl ammonium methylsulphate, disoya dimethyl ammonium chloride and dicoco dimethyl ammonium chloride; hair conditioning agents such as silicones, higher alcohols, cationic polymers and the like; enzyme stabilisers such as water soluble sources of calcium or borate species; colouring agents; TiO₂ and TiO₂-coated mica; perfumes and perfume solubilizers; and zeolites such as Valfour BV400 and derivatives thereof and Ca²⁺/Mg²⁺ sequestrants such as polycarboxylates, amino polycarboxylates, polyphosphonates, amino polyphosphonates etc. and water softening agents such as sodium citrate. Other optional materials include anti-dandruff actives such as ZPT, and perfumes.

The invention will now be illustrated with reference to the following example. In this example various standard tests are used as follows:

I Assessment of Initial Colour and Colour Change (Measurement of ΔE)

The equipment used to measure both the initial colour and colour change of substrates (hair/skin) dyed with the low pH colouring compositions of the present invention is a Hunter Colourquest spectrophotometer. The value used to express the degree of colour change on any particular substrate is Delta E (ΔE). Delta E, as defined herein, is represented by a factual sum of L, a, and b values such that:

ΔE=(ΔL²+Δa²+Δb²)^{½}

and L is measure of lightness and darkness (colour intensity), wherein L=100 is equivalent to white, and L=0 is equivalent to black. Further, “a” is a measure of the red and green quotients (colour hues) such that positive equates to red and negative to green and “b” is a measure of the yellow and blue quotients (colour hues) such that positive equates to yellow and negative equates to blue.

Hunter Colourquest measurements can be carried out on the Hunter Labscan Colourimeter which is a full scanning spectrocolorimeter with a wavelength of from 400-700 nanometers which records the colour of test hair switches (tresses) in terms of “L”, “a” and “b” values. The machine is set to: mode—0/45; port size—1 inch; view size—1 inch; light—D65; field of view—10°; UV lamp/filter—none. The hair is placed in a sample holder designed to hold the hair in uniform orientation during measurement. Equivalent colorimeters can be used, but it must be ensured that the hair does not move during measurement. The hair must be spread to cover the 1 inch port during colour measurement. Dots are placed on the switch holder to guide the positioning of the holder at the port. The dots are lined up with a mark on the port and readings are taken at each spot.

Eight measurements are run per switch, 4 on each side, and three switches are run per treatment.

II Standard Hair Switch

The compositions according to the present invention can be used to colour hair of all colours, types and condition. For the purposes of illustration various test hair switches can be tested herein. Two of these standard hair switches can be measured in terms of their approximate L, a, b values.

| | L | a | b |
|---|---|---|---|
| Light brown | about 60 | about 9 | about 32 |
| (permed and bleached) | | | |
| 40% grey dark brown | 35-37 | 4.5-5.5 | 11.5-12.7 |

Yak hair (virgin or permed and/or bleached) can also be used. It has values of: L=about 82 to 83, a=about −0.5 to 0.7, b=about 11 to 12.

III Hair Switch Colouring Method

To colour hair, a 4 gramme switch of about 8 inch long hair (or a 2 gramme switch of 4 inch long hair) is hung over a suitable container. The test colouring product is then prepared (ie, where applicable the separate bottle components are mixed together) and about 2 grammes of product per gramme hair is applied directly to the test hair switch. The colourant is massaged through the hair switch for up to about 1 minute and then left on the hair switch for up to about 30 minutes. After rinsing with running water for about 1 or 2 minutes the coloured hair switch is then cleansed (according to the shampoo protocol IV below) and dried. Drying can be effected either naturally (without heat assistance) or using a drier. The colour development (initial colour) of the coloured, cleansed, dried test hair switch can then be assessed using the Hunter Colourquest spectrophotometer.

For the delivery of a red shade (hue) to prepermed, prebleached light brown hair (having L, a, b values of approximately 60, 9 and 32) the preferred initial shade of the coloured hair will have a hue value (arc tangent of (b/a)) in the range of from about 25 to about 70, more preferably from about 30 to about 65, most preferably from about 35 to about 60 and wherein the initial colour intensity (L) is greater than about 10 and less than about 70, preferably greater than about 15 and less than about 65 more preferably greater than about 20 and less than about 60.

For the delivery of a brown or black shade (hue) to prepermed, prebleached light brown hair (having L, a, b values of approximately 60, 9 and 32) the preferred initial shade of the coloured hair will have a hue value (arc tangent of (b/a)) of less than about 25, preferably less than about 20 and the initial colour intensity (L) will be greater than about 1 and less than about 50, preferably greater than about 5 and less than about 45.

For the delivery of a light brown shade (hue) to prepermed, prebleached light brown hair (having L, a, b values of approximately 60, 9 and 32) the preferred initial shade of the coloured hair will have a hue value (arc tangent of (b/a)) in the range of from about 70 up to about 110 and wherein the initial colour intensity (L) will be greater than about 20 and less than about 95, preferably greater than about 25 and less than about 90.

A significant colour change, as delivered via the colouring compositions according to the present invention often means a colour change on permed and bleached hair in terms of Delta E which is preferably greater than about 5 or 8, preferably greater than about 10, more preferably greater than about 12, most preferably greater than about 15 and especially greater than about 20.

IV Hair Switch Cleansing Method

Switches of coloured hair are subjected to a repeated cleansing cycle wherein the following process is repeated.

A 4 gramme, 8 inch test switch (or a 2 gramme, 4 inch test switch) of coloured hair is clamped over a suitable container and rinsed thoroughly for about 10 seconds using warm water (at about 100° F. at about 1.5 gallons/minute pressure). Shampoo (about 0.1 ml non-conditioning shampoo per gramme hair) can then be applied directly to the wet test switch using a syringe. After lathering the hair for about 30 seconds the hair is rinsed in running water for about 30 seconds. The shampoo and lathering process is then repeated with a final 60 second rinse. Excess water can be removed (squeezed) from the test switch using the fingers. The test switch is then dried either naturally, or using a pre-heated dryer box at about 140° F. (for about 30 minutes). The coloured, cleansed, dried test hair switch can then be colour assessed (Delta E fade).

During any single test cycle each different switch to be assessed should be tested in water at equivalent temperature, pressure level and hardness level.

### EXAMPLE 1

In this example formulation 1 according to the invention is compared with a commercially available product and the two dyeing compositions compared for colour fading, measured by ΔE.

The compounds used are as follows:

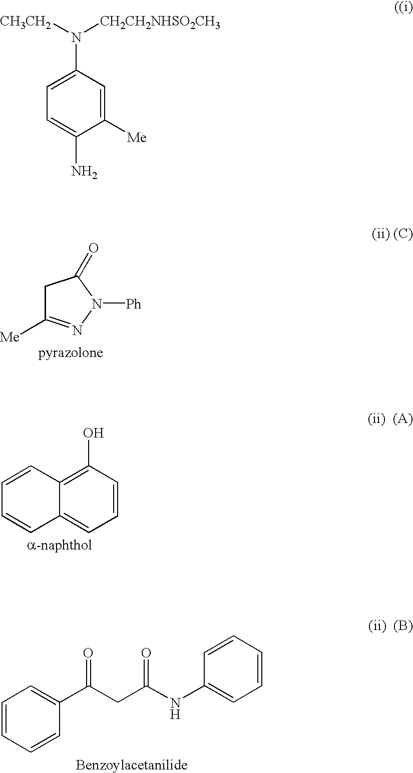

Formulation 1 had the following composition:

| | % by weight |
|---|---|
| Ceteareth 25 | 0.84 |
| Cetyl alcohol | 1.16 |
| Stearyl alcohol | 1.16 |
| (i) | 0.87 |
| Pyrazolone (i) (C) | 0.16 |
| α-naphtho1 (ii) (A) | 0.0046 |
| Benzoylacetanilide (ii) (B) | 1.86 |
| Ethanol | 9.3 |
| NH₄OH | 1.6 |
| Hydrogen Peroxide | 3.0 |
| Sodium Sulphite | 0.46 |
| EDTA | 0.46 |
| Water | up to 100 |

The pH of formulation 1 was from 9 to 10.

The comparative formulation was L'Oreal Recital “Santiago”.

8 grams of each formulation was applied to a 4 gram switch of light brown permed and bleached hair for 30 minutes as described in the colouring method above. Perming and bleaching were carried out according to the protocols described above.

Values of L, a and b were calculated on the undyed hair and the dyed hair. The difference in colour between the two states was calculated to give ΔE initial, in the manner described above for measuring ΔE.

The dyed switch was then subjected to 40 washes, using the washing protocol described above. After 40 washes the values of L, a and b were again measured and the difference in colour between the initial dyed switch and the washed switch was calculated as described above for ΔE, to give ΔE fade.

Values of ΔE initial, ΔE fade % fade are given below.

| | | | | ΔE | ΔE | |
|---|---|---|---|---|---|---|
| | L | a | b | initial | fade | % fade |
| Formula I | | | | | | |
| Before | 60.15 | 9.94 | 32.08 | | | |
| Dyed | 29.2 | 13.55 | 13.89 | 36.1 | | |
| 40 | 30.34 | 13.24 | 13.50 | | 1.25 | 3.5 |
| washes | | | | | | |
| L'Oreal | | | | | | |
| Before | 58.95 | 9.68 | 31.63 | | | |
| Dyed | 30.06 | 13.66 | 14.68 | 33.8 | | |
| 40 | 36.69 | 8.76 | 18.21 | | 9.00 | 26.6 |
| washes | | | | | | |

These results show that the commercial formulation and Formula 1 of the invention gave a similar ΔE initial, with the formulation of the invention in fact giving a greater ΔE and consequently a greater change in colour upon dyeing. However it will then be seen that the ΔE fade after 40 washes is significantly lower for formula 1 of the invention than for the commercially available product.

### EXAMPLE 2

This example illustrates the changes in initial colour uptake as measured by ΔE initial and colour fade as measured by ΔE fade which are achieved when antioxidant is incorporated into the composition of the invention. Three formulations were tested. Formulations 6 and 7 of the invention contain as antioxidant sodium sulphite. Comparative formulation 5 does not contain antioxidant. The compositions have the formulations give below:

| | Formulation 5 | | |
|---|---|---|---|
| Component | (Comparative) | Formulation 6 | Formulation 7 |
| Ceteareth 25 | 1.03 | 1.03 | 1.03 |
| Cetyl Alcohol | 1.54 | 1.54 | 1.54 |
| Stearyl Alcohol | 1.54 | 1.54 | 1.54 |
| (i) | 1.00 | 1.00 | 1.00 |
| (ii) (C) | 0.25 | 0.25 | 0.25 |
| (ii) (B) | 0.74% | 0.74 | 0.74 |
| (ii) (A) | 0.0074% | 0.0074 | 0.0074 |
| Na₂SO₃ | 0% | 0.22% | 1.1% |
| EDTA | 0% | 0.22% | 1.1% |
| NH₄OH | 0.5% | 0.5% | 0.5% |
| EtOH | 6% | 6% | 6% |
| Water | up to 100% | 100% | 100% |
| pH | 9-10 | 9-10 | 9-10 |

Colouring was carried out on a 4 gram switch of permed and bleached light brown hair as described in Example 1. After 20 washes the ΔE fade value was measured. Values of ΔE initial and ΔE fade are given in Table 3 below.

| Formulation | ΔE Initial | ΔE Fade |
|---|---|---|
| 5 | 13.4 | 4.2 |
| 6 | 14.7 | 1.7 |
| 7 | 15.6 | 1.0 |

This shows that ΔE fade for formulation 5, which contains no antioxidant, is significantly greater than the ΔE fade for formulations 6 and 7. surprisingly, formulations 6 and 7 show improved initial colour uptake, as illustrated by greater ΔE initial.

### EXAMPLE 3

The comparisons below demonstrate the improved ΔE fade values which can be obtained using a preferred coupler (b). The formulations below were tested. The N,N-diethylacetoacetamide is a preferred yellow coupler (b) and p-carboxybenzylacetanilide is a yellow coupler used in GB 1,025,916. The formulations tested are as follows:

| Formula | I | II | III | IV |
|---|---|---|---|---|
| Cetyl alcohol | 1.4 | 1.4 | 1.4 | 1.4 |
| Stearyl alcohol | 1.4 | 1.4 | 1.4 | 1.4 |
| Ceteareth-25 | 1.0 | 1.0 | 1.0 | 1.0 |
| 4-(N-ethyl,N-hydroxyethyl-p- | 1.1 | 1.1 | 1.1 | 1.1 |
| phenylenediamine | | | | |
| p-Carboxybenzoylacetanilide | 2% | 3.6% | / | / |
| N,N-Diethylacetoacetamide | / | / | 2% | 2% |
| EDTA | / | / | / | 0.2% |
| Ascorbic acid | / | / | / | 0.4% |
| Ammonia | 1.4 | 1.4 | 1.4 | 1.4 |
| peroxide | 2.9 | 2.9 | 2.9 | 2.9 |
| Water | to 100 | to 100 | to 100 | to 100 |

Experimentation was carried out either on virgin (undamaged) yak hair or permed (damaged) yak hair. All formulae were applied to the relevant switch at room temperature for 30 minutes at pH about 10. Formulae I and III contain the two different couplers in a 1:1 wt ratio and formulae II and IV contain the two different couplers in a molar ratio of 1:1. Formulation IV contains ascorbic acid as antioxidant. Results were as follows:

| | | delta E | delta E fade 20 | |
|---|---|---|---|---|
| Switch | Formula | uptake | washes | Comments |
| virgin | I | 32 | 4.5 | light brown colour - poor |
| yak | | | | yellow uptake |
| virgin | II | 31 | 3.0 | poor yellow colour |
| yak | | | | |
| virgin | III | 41 | 1.5 | light yellow |
| yak | | | | |
| permed | I | 29 | 6.5 | light green yellow |
| yak | | | | |
| permed | II | 28 | 5.5 | light green yellow |
| yak | | | | |
| permed | IV | 48 | 2.5 | bright intense green yellow |
| yak | | | | |

It can be seen that the formulations III and IV containing the preferred coupler of the invention give ΔE fade values of 2.5 and 1.5. The other compositions on the other hand give higher ΔE fade values. On permed (damaged) hair formulation IV gives low ΔE fade of 2.5. Permed hair tends to fade more rapidly than undamaged hair in general.

### EXAMPLE 4

In this example two formulations were compared. One was a commercially available formulation “Santa Cruz”, available from L'Oreal. The other was formulation (A) of the invention, given below. The two formulations were applied for 30 minutes at pH about 9 to 10 to (a) virgin (undamaged) yak hair and to (b) permed (damaged) yak hair. Each type of hair had values of L=83, a=−0.4, b=11. The switches were coloured using the protocol discussed above, washed 20 times using the protocol discussed above and the value of ΔE fade measured.

Formulation A of the invention was as follows:

Formulation A

| | |
|---|---|
| Ceteareth-25 | 0.84% |
| Cetyl Alcohol | 1.16% |
| Stearyl Alcohol | 1.16% |
| 2,6-Dichloro-p-aminophenol | 0.87% |
| N,N-Dimethylacetoacetamide | 2.8% |
| 3-Methyl-pyrazolin-5-one | 0.64% |
| Asorbic Acid | 0.4% |
| n-Propyl gallate | 0.8% |
| EDTA | 0.2% |
| Ammonia | 1.4% |
| Peroxide | 3% |
| Water | up to 100% |

Results were as follows:

after dyeing

| Hair | Product | L | a | b | ΔE fade |
|---|---|---|---|---|---|
| (a) | Santa Cruz | 36 | 23 | 20 | 2.2 |
| (b) | Santa Cruz | 26 | 24 | 16 | 6.0 |
| (a) | A | 34 | 17 | 18 | 1.8 |
| (b) | A | 25 | 19 | 13 | 3.1 |

It can be seen that the fading from the undamaged hair (a) is lower with formulation A than with the commercially available product. In particular the fading with formulation A is significantly lower from the damaged hair (b) than with the commercially available product. With the commercially available product the difference in fading between damaged and undamaged hair 273% whereas for formulation A the difference is only 172%.

1. A hair colouring composition comprising
(i) one or more developers selected from amino aromatic systems capable of being oxidised and thereafter undergoing a single electrophilic attack, and
(ii) one or more couplers selected from
(A) phenols and naphthols having an active leaving group in the para position relative to the OH group,
(B) 1,3-diketones containing the group 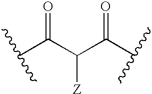
in which Z is an active leaving group, and
(C) compounds containing the group 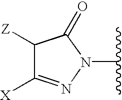
in which Z is an active leaving group, and X is an active leaving group or a non-leaving substituent,
such that in the presence of an oxidising agent the or each developer reacts with the or each coupler substantially only at the position having the active leaving group Z and, if X is an active leaving group, X,
which when applied to hair by the hair switch colouring method described herein and washed 20 times by the washing protocol described herein, gives a value of ΔE fade, measured as described herein, as follows:
(a) when the composition is suitable for delivering a blonde or light brown shade ΔE fade is not more than 2.5
(b) when the composition is suitable for delivering a red shade, ΔE fade is not more than 5.0
(c) when the composition is suitable for delivering a black or dark brown shade ΔE fade is not more than 2.5.

2. A composition according to claim 1 in which the one or more developers (i) are selected from N,N-disubstituted p-phenylene diamines.

3. A composition according to any preceding claim in which (a) when the composition is suitable for delivering a blonde or light brown shade ΔE fade is not more than 1.0.

4. A composition according to any preceding claim in which (b) when the composition is suitable for delivering a red shade, ΔE fade is not more than 1.8.

5. A composition according to any preceding claim in which (c) when the composition is suitable for delivering a black or dark brown shade, ΔE fade is not more than 1.0.

6. A composition according to any preceding claim which contains not more than 0.1 wt % oxidative colouring agents which are not of the types (i), (A), (B) and (C).

7. A composition according to any preceding claim which contains not more than 0.1 wt % oxidative colouring agents which are capable of undergoing reaction more than once under the conditions of hair colouring.

8. A composition according to any preceding claim which contains not more than 0.1 wt % of any oxidative colouring agent which can react with itself under the conditions of hair colouring.

9. A composition according to any preceding claim in which substantially all molecules of developer (i) do not react substantially with other developer molecules, under the conditions of hair colouring.

10. A composition according to any preceding claim which has a pH of at least 6.1, preferably at least 6.5.

11. A composition according to any preceding claim which contains ammonia or ammonium hydroxide in an amount of at least 0.01 wt %.

12. A composition according to any preceding claim in which each of the developers and couplers has a solubility at pH about 10 and 25° C. at least 10 g/100 ml deionised water.

13. A composition according to any preceding claim in which each of the final coloured dimers or trimers produced by reaction of the developer(s) with the coupler(s) at pH about 8 and 25° C. is not more than 5 g/100 ml deionised water.

14. A composition according to any preceding claim which gives ΔE fade on damaged hair not more than 200% of its ΔE fade on undamaged hair

15. A method of colouring hair comprising providing
(i) one or more developers selected from amino aromatic systems capable of being oxidised and thereafter undergoing a single electrophilic attack, and
(ii) one or more couplers selected from
(A) phenols and naphthols having an active leaving group in the para position, relative to the hydroxyl group,
(B) 1,3-diketones containing the group 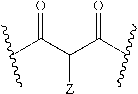
in which Z is an active leaving group, and
(C) compounds containing the group 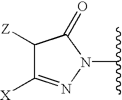
in which 2 is an active leaving group, and X is an active leaving group or a non-leaving substituent,
such that in the presence of an oxidising agent the or each developer reacts with the or each coupler substantially only at the positions having the active leaving group Z and, if X is an active leaving group, X,
and (iii) oxidising agent, and applying them to the hair,
and in which the mixture of materials is such that when applied to hair by the hair switch colouring method described herein and washed 20 times by the washing protocol described herein, gives a value of ΔE fade, measured as described herein, as follows:
(a) when the composition is suitable for delivering a blonde or light brown shade, ΔE fade is not more than 2.5
(b) when the composition is suitable for delivering a red shade, ΔE fade is not more than 5.0,
(c) when the composition is suitable for delivering a black or dark brown shade, ΔE fade is not more than 2.5.

16. A method according to claim 15 in which the hair is substantially undamaged.

17. A method according to claim 15 in which the hair has been bleached, permed and/or previously coloured.

18. A method according to claim 15 in which part of the hair is substantially undamaged and part of the hair has been previously bleached, permed and/or previously coloured.
